SEQUENCE LISTING (29 sequences)

SEQ ID NO: 1 - protein, 377 aa, Gluconacetobacter diazotrophicus
MTQPLLRTPLHDLHLSLGARMVPFGGYEMPVQYRAGVMAEHQHTRAAAGLFDVSHMGQIRLRARSGRVEDAALALERLVPADILSLKPGRQRYALLTNEQGGIIDDLMVSRVGDTLLLVVNAACKDADLAHITAALDDACIVESLPDRALIALQGPLAGAALARLAPASADMRFMDVAEFDVAGVPCIVSRSGYTGEDGFELGMESGGTVRVAEALLAQAEVEPVGLGARDSLRLEAGLCLYGSDIGPDTTPVEGALEWSIQKSRRAGGARAGGFPGADIILARIQDGAARRRVGIAVDGRAPVRGGARLFADAEGHRPVGHVTSGAFGPTAGAPVAMGYVDIAHAATGTALFAELRGKYVPVTVAALPFVAPGFKR

SEQ ID NO: 2 - protein, 702 aa, Gluconacetobacter diazotrophicus
MDVKRAWSGDPLQPGDRIVADFRSLYRHGFARVAGCTLPVALADPATNVARMADMVRACHADGVALAVFPELGVSGYTIEDLRQQDVLLDGVGAALAALAAATADLVPVVVAGAPLRHGDALYNCAVILHRGTVLGVVPKSYLPNYREFYEARQFAPGAGLRGQTIHVAGQTAPFGTDLLFEAEDVPGLVIAIEICEDLWVPAPPSTDAALAGATVIANPSASDITVGKADTRDLLCRSQSARALCAYVYAAAGEGESTTDLAWDGQVSVYENGALLAETVRFPQGPNRATADIDLDLLRQERARMGSFADNRAARGLHATGGDTGWRRVGFALAPPPGDLGLMRRIERFPFVPADPARLEQDCYEAWTIQVSALKQRLQATGTRRMVIGVSGGLDSTQALLVAVRAADELGLGRDAVLAYTMPGFGTSAGTQSNAMALMQALGVTAAELDIRPTARMMLEQMGHPFASGVPQYDVTFENVQAGLRTDFLFRLANQHGGIVIGTGDLSELALGWCTYGVGDQMSHYNVNAGLPKTLIQHLIRWVISARRVDDDAAGVLASILDTEISPELVPAGEDQALQSTEERIGPYALQDFTLFYVLRHGFRPSRIAFMAEIAWKDAGIGAWPPGFPADRRVEYDLPTIRHWLSVFLTRFFGFSQFKRSAMPNGPKVVAGGALSPRGDWRAPSDGNARLWLEELERNVP

SEQ ID NO: 3 - protein, 515 aa, Gluconacetobacter diazotrophicus
MMTDSLILTPGTLSLADLRRLAFSAPSVTLAAGVRDTLAQAARSVDRIVADGRPVYGVNTGFGKLARTRIADANLRDLQRNLVLSHAAGIGAPMDDRTVRLILLLKANGLARGHSGVRPEIVDLLLEMGNRGVLPVIPQKGSVGASGDLAPLAHMTAVLIGAGQARVDGRVLPGDQALRAVGLAPVELGPKEGLALLNGTQASTALALVALFDAERVFQAALVTGALTLDAARGTDAPFDPRLHALRGQKGQIECAALYRALMADSAIRASHREDDERVQDPYCLRCQPQVMGACLDSLRHAASVLLIEANAVSDNPIHFAETDEMLSGGNFHAEPVAIAADLMAIAVSEVGAIAERRLALLVDAQMSGLPPFLVRDSGVNSGFMIAQVTAAALASENKTLAHPASIDSLPTSANQEDHVSMATFAARRVGDIVANVRDIVAIEYLAAVQGLDFLAPLQTSRPLAGAAAALRARVPFYDRDRIFTPDIEAARDLIADGVPTLLPGIADVLPRLEV

SEQ ID NO: 4 - protein, 296 aa, Gluconacetobacter diazotrophicus
MDLKSLEAVLWIDRLGGFKAAAEALRMTQPAISIRVSQLEAMLETRIFHRSGKRVVPTPVGVTLIHYAERILQLRDEALLTIKGHNEENGILRLGTVETTAHTWLAQLLCRIDEKYPNITVDLDIGISDDIQRKVAKGMLDVGLFMGPVNSPMMIETPVCSFEVALLAHRTVVGQRGGRTDLGSLPALPIMTFSRNTVPHSMLHRLLGREGGSHHRVHAMSSVPTIVSMLLSGAGLALLPPDVVREHLDSGALCRLDVGQALPPLNCVAGRLMKYSSGLVEDVVAMARNVAQSAGA

SEQ ID NO: 5 - protein, 412 aa, Gluconacetobacter diazotrophicus
MLVSTALVLMMTVPGLALFYGGMVRKKNVLATLMQSFAICCIVTIVWMVAGYSLAFGTGSPYIGDLSRFMLNGIGAQISKGSDVGFTLGLGSANATVMTIPESVFMMFQMTFAIITPALITGAFAERMKFSALCVFTILWSLLVYAPVAHWVWSPLGWVAGFGAIDFAGGTVVHINAGVAGLVTALVVGKRQGYGQDDMSPFNLTYAVIGASLLWVGWFGFNAGSAVGANGRAGMAMATTQIASAAAGVAWMLAEWARTGKPTVLGIISGAVGGLVAITPAAGFVLPGGALIIGLLAGAVCYWTATTMKHMLGYDDSLDAFGVHGIGGILGALLTGVLAYGPLSATDANPAGVVGSFAQLVTQAKAVGVTIVWCGVVTFILLKIVDLAIGLRVRSEDEIEGLDMTQHGERIN

SEQ ID NO: 6 - protein, 412 aa, Gluconacetobacter diazotrophicus
MLTSTALVLMMTVPGLALFYGGMVRKKNVLATLMQSFAICCIITVLWMVAGYSLTFGTGSPYIGDLSRFMLNGIGAQISKGSDVGFTLGLGSANATVMTIPESVFMMFQMTFAIITPALIAGSFAERMKFSALCVFTILWSLLVYAPIAHWVWSPLGWVAGFGAVDFAGGTVVHINAGIAGLVTALVLGKRQGYGQDDMSPFNLTYAVIGASLLWVGWFGFNAGSAVGSNGRAGMAMATTQIATAAAGLSWMLAEWARTGKPTVLGIISGAVAGLVAITPAAGFVLPGGALVIGLITGAVCYFAATSLKHMLGYDDSLDAFGVHGIGGILGALLTGVLAYGPLSATDANPAGVVGSFAQFVTQAKAVGVTIVWCGVVTFILLKIVDLAIGLRVTTEQEQQGLDMSLHGEKIS

SEQ ID NO: 7 - protein, 418 aa, Gluconacetobacter diazotrophicus
MKVIVLGAGVIGVTSAWYLAKLGHEVEVVDRQPAAAMETSFANAGQVSPGYSTPWAMPGLPRKALGWMLQKHSPLVIRARIDFAMFRWMTQLLTNCTEHAYDVNKARMLRIAEYSRDCLTALREETGITYDDRQRGLIQLFRTDAQLEHAHEDMRLLAESEVPHELLDVAAIVRREPGLAHAQHLLKGGLFLPGDESGDAHMFTQRLAQKAEELGVTFHYETGIEGLDASASEILGVRTSTGRMTGDAYVVALGSYSPLLLRPMGIRLPVYPVKGYSLTVPLTDPDRAPVSTVNDETYKVAMTRLGDRIRIGGTAELTGYDLRLSPDRRETLELSFSDLFGGGDLGRATYWTGLRPNTPDGTPVVGPSGRFRNLWLNTGHGTLGWTMACGSGHMLADLIAGRRPNIPHLDLSIDRYAS

SEQ ID NO: 8 - DNA, 25 bp, synthetic construct
  source 1..25: mol_type = other DNA
cacatgttctttcctgcgttatccc

SEQ ID NO: 9 - DNA, 23 bp, synthetic construct
  source 1..23: mol_type = other DNA
cgcctagcttcctgctgaacatc

SEQ ID NO: 10 - DNA, 32 bp, synthetic construct
  source 1..32: mol_type = other DNA
nnngaattcgtgctgccagcccataaatcagg

SEQ ID NO: 11 - DNA, 36 bp, synthetic construct
  source 1..36: mol_type = other DNA
nnntctagacgactcgctggaatatttgggcgacac

SEQ ID NO: 12 - DNA, 44 bp, synthetic construct
  source 1..44: mol_type = other DNA
nnnggatccgaattctgtgcatatggcgtcttctcccgtctcgc

SEQ ID NO: 13 - DNA, 38 bp, synthetic construct
  source 1..38: mol_type = other DNA
nnnggatccctgatccatttcaccgaacaacagacagg

SEQ ID NO: 14 - DNA, 45 bp, synthetic construct
  source 1..45: mol_type = other DNA
nnngaattccttttttatttcacaatcgatgcaaccagcgtttcc

SEQ ID NO: 15 - DNA, 36 bp, synthetic construct
  source 1..36: mol_type = other DNA
nnnaagcttcttcgtcagccatggcaggtgggtatc

SEQ ID NO: 16 - DNA, 55 bp, synthetic construct
  source 1..55: mol_type = other DNA
nnnggatccgaattcgttcatatggcaatctccccggtttcattcgtacggatac

SEQ ID NO: 17 - DNA, 29 bp, synthetic construct
  source 1..29: mol_type = other DNA
nnnggatcctgatcgttccggaaacgggc

SEQ ID NO: 18 - DNA, 25 bp, synthetic construct
  source 1..25: mol_type = other DNA
gatcgtctcatgggcactcagtgac

SEQ ID NO: 19 - DNA, 24 bp, synthetic construct
  source 1..24: mol_type = other DNA
gtgtatcagaccgttccgctgcag

SEQ ID NO: 20 - DNA, 30 bp, synthetic construct
  source 1..30: mol_type = other DNA
gaatgacgagacgtacaaggtcgccatgac

SEQ ID NO: 21 - DNA, 28 bp, synthetic construct
  source 1..28: mol_type = other DNA
cgatagatttcgccgcgataattgaagg

SEQ ID NO: 22 - DNA, 34 bp, synthetic construct
  source 1..34: mol_type = other DNA
nnntctagactggtgaggtggaggaggcaagtag

SEQ ID NO: 23 - DNA, 25 bp, synthetic construct
  source 1..25: mol_type = other DNA
ggtcagcgacgtccggttggtttcg

SEQ ID NO: 24 - DNA, 24 bp, synthetic construct
  source 1..24: mol_type = other DNA
caggttttcccggtcccgctgcac

SEQ ID NO: 25 - DNA, 20 bp, synthetic construct
  source 1..20: mol_type = other DNA
gcgccggttgccgatggcgg

SEQ ID NO: 26 - DNA, 33 bp, synthetic construct
  source 1..33: mol_type = other DNA
nnnggatcccaggttttcccggtcccgctgcac

SEQ ID NO: 27 - DNA, 29 bp, synthetic construct
  source 1..29: mol_type = other DNA
nnnggatccgcgccggttgccgatggcgg

SEQ ID NO: 28 - protein, 20 aa, synthetic construct
DRENLLHDSGLAQPAAPVAD

SEQ ID NO: 29 - protein, 10 aa, Gluconacetobacter diazotrophicus
DRENLAPVAD

(19) United States
(12) Patent Application Publication
(10) Pub. No.: US 2026/0176644 A1
(43) Pub. Date: Jun. 25, 2026

(54) GENETICALLY MODIFIED ENDOPHYTIC DIAZOTROPHS AND METHODS OF USING SAME

(71) Applicant: REGENTS OF THE UNIVERSITY OF MINNESOTA, Minneapolis, MN (US)

(72) Inventors: Brett Barney, Minneapolis, MN (US); Benjamin R. Dietz, Minneapolis, MN (US); Neil E. Olszewski, Minneapolis, MN (US)

(21) Appl. No.: 19/129,849

(22) PCT Filed: Nov. 9, 2023
(86) PCT No.: PCT/US2023/079189

§ 371 Date: May 14, 2025

Related U.S. Application Data
(60) Provisional application No. 63/425,288, filed on Nov. 14, 2022.

(51) Int. Cl. C12N 15/82; C12N 1/20; C12N 1/205; C12N 9/80; C12N 15/74

(52) U.S. Cl. CPC C12N 15/8261; C12N 1/20; C12N 1/205; C12N 9/80; C12N 15/74

(57) ABSTRACT

A diazotrophic microbe is genetically modified to secrete a nitrogen-containing compound in an amount greater than a comparable control diazotrophic microbe. In one or more embodiments, the diazotrophic microbe is an endophyte. In one or more embodiments, the genetic modification includes a deletion or disruption of a gene involved in import or export of nitrogen-containing compounds. In some of these embodiments, the genetic modification includes a deletion or disruption of a gene encoding a gene product that transports ammonium into the cell, recycles lost ammonium back into the cell, and/or transports ammonia out of the cell. In one or more embodiments, the amount of secreted nitrogen-containing compound is effective to support the growth of a non-diazotroph in co-culture.

### CROSS-REFERENCE TO RELATED APPLICATION

This application claims the benefit of U.S. Provisional Patent Application No. 63/425,288, filed Nov. 14, 2022, which is incorporated herein by reference in its entirety.

### GOVERNMENT FUNDING

This invention was made with government support under 2020-67019-31148 awarded by the National Institute of Food and Agriculture. The government has certain rights in the invention.

### SEQUENCE LISTING

This application contains a Sequence Listing electronically submitted via Patent Center to the United States Patent and Trademark Office as an .xml file entitled “0110-000704WO01.xml” having a size of 30 kilobytes and created on Nov. 9, 2023. The information contained in the Sequence Listing is incorporated by reference herein.

### SUMMARY

This disclosure describes, in one aspect, a diazotrophic microbe genetically modified to secrete a nitrogen-containing compound in an amount greater than a comparable control diazotrophic microbe.

In one or more embodiments, the microbe does not include an undesirable exogenous gene.

In one or more embodiments, the microbe does not include a selection marker.

In one or more embodiments, the genetic modification is a deletion or disruption of a gene involved in import or export of nitrogen-containing compounds. In one or more of these embodiments, the genetic modification includes a deletion or disruption of a gene encoding a gene product that transports ammonium into the cell, recycles lost ammonium back into the cell, and/or transports ammonia out of the cell.

In one or more embodiments, the genetic modification includes a deletion or disruption of at least a portion of a gene homologous to G. diazotrophicus amtB. In one or more of these embodiments, the genetic modification is a deletion or disruption of a gene homologous to Gdia_0598 and/or Gdia_1302 of G. diazotrophicus. In one or more of these embodiments, the genetic modification includes a deletion or disruption of a Q-linker region in G. diazotrophicus amtB or a gene homologous to G. diazotrophicus amtB.

In one or more embodiments, the genetic modification is a deletion or disruption of at least a portion of a gene homologous to G. diazotrophicus NifA. In one or more of these embodiments, the genetic modification includes a deletion or disruption of a Q-linker region in G. diazotrophicus NifA or a gene homologous to G. diazotrophicus NifA.

In one or more embodiments, the genetic modification includes a clean deletion.

In one or more embodiments, the amount of secreted nitrogen-containing compound is effective to support the growth of a non-diazotroph in co-culture.

In one or more embodiments, the nitrogen-containing compound includes ammonia or ammonium.

In one or more embodiments, the microbe is derived from a bacterium of the class Alphaprotobacteria.

In one or more embodiments, the microbe is derived from a bacterium of the phylum Pseudomonadota.

In one or more embodiments, the microbe is derived from Gluconacetobacter diazotrophicus.

In one or more embodiments, the microbe is derived from Pseudomonas stutzeri, Rhodobacter sphaeroides, Azospirillum sp., Azoarcus sp., Herbaspirillum sp., Klebsiella sp., or Burkholderia sp.

In one or more embodiments, the microbe fixes nitrogen micro-aerobically.

In one or more embodiments, the microbe fixes nitrogen anaerobically.

In one or more embodiments, the microbe fixes nitrogen aerobically.

In another aspect, this disclosure describes a method of increasing growth of an organism. Generally, the method includes co-culturing the organism and any embodiment of a diazotrophic microbe genetically modified to secrete a nitrogen-containing compound in an amount greater than a comparable control diazotrophic microbe.

In one or more embodiments, the co-culture method increases the growth rate of the organism.

In one or more embodiments, the co-culture method increases the cell density of the organism.

In one or more embodiments, the co-culture method increases the crop yield of the organism.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Plasmid constructs. (A) Agarose gel displaying conformation of amtB deleted genes. Lanes 1 and 2: Wild-type G. diazotrophicus with unmodified amtB genes. Lanes 3 and 4: G. diazotrophicus with deletion of amtB1 and no modification of amtB2. Lanes 5 and 6: G. diazotrophicus with deletion of both amtb1 and amtb2. Lane 7:1-kilobase DNA ladder. (B) Annotated plasmid maps of the four final plasmid constructs. Plasmids pGAMTB1 and pGAMTB2 are used to delete amtB1. pGAMTB3 and pGAMTB4 are used to delete amtB2.

FIG. 2. In vitro culture experiments. (A) Co-culture growth on medium without nitrogen. The two spots on the left are G. diazotrophicus wild-type grown with C. sorokiniana. The two spots on the right are the dual amtB deletion strain of G. diazotrophicus grown with C. sorokiniana. The green color indicates increased nitrogen production and/or release by the dual amtB strain of G. diazotrophicus as compared to wild-type G. diazotrophicus. (B) A culture plate showing blue/white selection. Example showing how after recombination of the flanks white colonies can be screened quickly and cleaned for conformation of clean deletion.

FIG. 3. The primary nitrogen fixation cluster of G. diazotrophicus. (A) Gene fitness values for the two growth conditions. Closed circles are without nitrogen grown under micro-aerobic conditions (N−) while open circles are with nitrogen provided as ammonium (N+). Genes were color-coded based on the N-fitness values. (B) A gene map of the primary nitrogen fixation cluster.

FIG. 4. Additional nitrogen assimilation and regulation genes of G. diazotrophicus. (A) shows gene fitness values for the two growth conditions. Closed circles are without nitrogen grown under micro-aerobic conditions (N−) while open circles are with nitrogen provided as ammonium (N+). Genes were color-coded based on the N-fitness values. (B) Gene clusters of G. diazotrophicus. Dotted lines represent large gaps between genes.

FIG. 5. Differential fitness analysis of G. diazotrophicus genes. (A) Additional genes showing differential fitness values during diazotrophic growth for G. diazotrophicus. Shown above are a selection of genes with minimal fitness defects during growth with supplemented nitrogen (N+, open circles) versus significant growth defects when grown without provided nitrogen under micro-aerobic conditions (N−, closed circles). (B) Genes displaying improved differential fitness values during diazotrophic growth for G. diazotrophicus. Shown above are a selection of genes with minimal fitness defects during growth with supplemented nitrogen (N+, open circles) versus significant growth improvements when grown without provided nitrogen under micro-aerobic conditions (N−, closed circles). Genes were selected that showed significant improvement and low variability. A separate data set for growth on rich medium (GAD) is also provided as reference.

FIG. 6. Shown is an illustration of the progression taken to manipulate the genome of Gluconacetobacter diazotrophicus. In the first step, a section of the genome (amtB1) is targeted for gene deletion. Flanking segments F1 and F2 of this gene were cloned into restriction sites (RE) of pPCRERIN7 (bottom left). In step one, the tetracycline (Tet^{R}) selection and lacZ (blue color identification) cassette from pLACZF19 was inserted between the flanks of pPCRERIN7 to make pGAMTB1. This plasmid was transformed into G. diazotrophicus and double homologous recombinants were selected. Next, pGAMTB2 was constructed by inserting the kanamycin (Kan^{R}) selection and sacB (conditionally toxic with sucrose) cassette from pPCRSACB28 into the restriction enzyme site (RE) adjacent to F2. This plasmid was transformed into the strain with the Tet^{R}/lacZ cassette and single homologous co-integrants were selected. There are two potential recombination products depending upon if pGAMTB2 has recombined with F1 (Option 1) or F2 (Option 2). Strains produced in Step 2 were allowed to grow without antibiotic pressure for two days to allow flanking regions to recombine, and then subjected to sucrose toxicity pressure to select for strains that had lost the sacB gene. Undesired recombination events are shown in red, while desired recombination events are shown in blue. The desired trait can be selected at this point by growth with X-Gal to look for strains that no longer harbor the lacZ gene and the resulting blue phenotype.

FIG. 7. An exemplary micro-aerobic chamber with supplemented LED lights used to co-culture G. diazotrophicus strains and C. sorokiniana. Lights were turned on and off on an eight hour cycle.

FIG. 8. Images of plates of Chlorella sorokiniana grown together with wild-type and manipulated strains of Gluconacetobacter diazotrophicus. (A) Selected strains following 10 days of growth. (B) Selected strains following three weeks of growth.

FIG. 9. Ammonium accumulation over time during aerobic diazotrophic growth of various Gluconacetobacter diazotrophicus strains. G. diazotrophicus strains were grown in Erlenmeyer flasks following transfer of planktonic cells to a medium containing limited asparagine (1.25 mM). The upper plot shows ammonium levels obtained at different time points. The lower plot shows the corresponding OD₆₀₀ plotted on a Log₂ scale. Cultures were grown at 180 rpm and 28° C. under a standard atmosphere. Results represent the average and standard deviation for at least three samples. Sampling times were offset by 0.5 hours in certain cases to better illustrate the standard deviation for individual strains. Cultures remained in planktonic growth through 72 hours, but GABB034 and GABB040 began to show signs of aggregation at 96 hours.

### DETAILED DESCRIPTION OF ILLUSTRATIVE EMBODIMENTS

This disclosure describes several tools involving genetically modified diazotrophic microbes to enhance nitrogen fixation and delivery to organisms in co-culture. In one aspect, the diazotrophic microbe is an endophyte. In another aspect, the diazotrophic microbe has genetic deletions. These genetic deletions may result in increased secretion of fixed nitrogen.

Fertilizer inputs from industrial processes such as the Haber-Bosch process come at the expense of fossil fuels. Nutrient requirements are directly linked to biomass production, and any potential increased improvement in the scale of biomass yield will necessitate a proportional increase in the demand for essential nutrients. For all photosynthetic systems—e.g., photoautotrophs such as land plants, algae, and cyanobacteria—with requisite light energy and water, nitrogen is a limiting and expensive nutrient input for aquaculture and agricultural production alike. Current nitrogen fertilizer production involves burning of fossil fuels to generate ammonia from molecular nitrogen (N₂ gas) through the Haber-Bosch process, is extremely energy intensive. In developed countries, industrial nitrogen production is accompanied by a huge overall economic and energetic cost. In developing countries, lack of nitrogen often limits agricultural productivity, where energy and infrastructure costs impede the use of the Haber-Bosch process to produce ammonia on a large scale from atmospheric nitrogen.

Many organisms naturally fix atmospheric nitrogen into forms that can be used as fertilizer. As used herein, “nitrogen fixation” and “nitrogen assimilation” refer to the process of incorporating nitrogen from a source that is not typically useable as a fertilizer, such as atmospheric nitrogen, into a form that can be used as a fertilizer. Organisms that fix nitrogen and their byproducts are referred to as “biofertilizers”. The development of improved biofertilizers represents a unique opportunity to lower the potential economic costs and environmental impacts of current fossil-fuel-dependent industrial methods for producing ammonia-derived fertilizers.

Current biofuel feedstock crops such as corn for ethanol require substantial amounts of nitrogen inputs. Potential future production of biomass using next generation feedstocks such as algae promise significant improvements in overall yield that could be orders of magnitude higher than current conventional land plant crops. Since current nitrogen requirements for the growth of biofuel crops are derived from energy intensive industrial processes such as Haber-Bosch, the energy use efficiency of current biofuel crops has been questioned. A significant amount of the energy acquired from, for example, corn ethanol, soybean biodiesel, and/or next generation biofuel crops (e.g., algae) may need to be diverted back to these industrial processes to supply the energy required for additional industrial nitrogen fixation. Improvements in final biomass yield can involve concomitant increases in macronutrient inputs such as nitrogen. Thus, the impacts and requirements of current methods to provide nitrogen for current and future crops will only increase in importance.

The approach described herein can circumvent the energy cost and the associated greenhouse gas emissions tied to producing and distributing nitrogen fertilizers by using an endophytic diazotrophic bacterium as a biofertilizer to provide a renewable source of nitrogen to meet the growth requirements of the associated photosynthetic species. As used herein, an “endophyte” or an “endophytic” organism has a symbiotic relationship with an associated organism, typically a photosynthetic organism such as a plant. While model symbiotic systems between specific plants and nitrogen-fixing bacteria are well established, these are limited to a small number of commodity crops. The approach described herein is directed towards expanding similar symbiotic relationships to a broader range of crops or next-generation biomass sources.

This disclosure describes compositions and methods involving genetically-modified diazotrophic microbes to enhance nitrogen fixation, and, therefore, support the growth of other organisms in co-culture. In one or more embodiments, the microbes described herein are genetically modified to secrete nitrogen-containing compounds at higher levels as compared to non-genetically modified microbes. As used herein, nitrogen-containing compounds “secreted” by an organism may pass from the organism to the external environment in any manner. “Secreted” compounds may be actively transported, passively diffused, or otherwise moved from the organism to the external environment. In one or more embodiments, higher levels of nitrogen-containing compounds in the soil are, in part, a result of decreased transport of nitrogen-containing compounds into an organism from the external environment.

In one aspect, this disclosure describes a genetically-modified strain of a nitrogen-fixing microbe. In one or more embodiments, the nitrogen-fixing microbe is a diazotroph, As used herein, a “diazotroph” is a microbe capable of fixing nitrogen from the atmosphere into a usable form of nitrogen, such as ammonia. Diazotrophs can be naturally found free-living or in association with other organisms, such as plants. Free-living diazotrophs typically live in the soil and secrete fixed nitrogen into the surrounding soil. Plants living in the soil can acquire and use this nitrogen. However, nitrogen secreted into the soil is susceptible to loss and may be inconsistently used by plants.

Diazotrophs that live within or in associate with other organisms without causing apparent disease are typically referred to as endophytes. Some endophytes are known to benefit the associated organism, while others are poorly understood. Endophytes may be bacteria or fungi. Endophytes frequently transfer nitrogen to associated organisms with greater efficiency than free-living diazotrophs. In one or more embodiments, the microbe described herein is an endophyte.

In one or more embodiments wherein the microbe is an endophyte, the associated organism is a plant. The plant may be a vascular plant, such as a legume, tree, or grass. The plant may alternately be photosynthetic organism, such as algae. The associated organism may be a biofuel feed crop, such as corn, wheat, soy, algae, sugarcane, sugar beet, sorghum, grasses, or canola. The plant may be grown on any suitable scale and using any suitable method, such as soil-based growth, hydroponic growth, or any other growing system.

In one or more embodiments, the microbe may not require formation of a specialized structure, such as a root nodule, to grow symbiotically with an organism. Some diazotrophic microbes grow within the root nodules of other organisms, such as plants. However, many plants and other organisms that require nitrogen do not form root nodules or other structures to promote colonization by diazotrophic microbes. Microbes that can grow in co-culture with other plants without specialized structures may be more adaptable to co-culture with plants that may not otherwise be colonized by diazotrophic microbes.

Many diazotrophic microbes may be compatible with the compositions and methods described herein. As used herein, a microbe “derived from” a naturally-occurring microbe is analogous to the referenced naturally-occurring microbe with one or more genetic modifications. Thus, the resultant microbe is not identical to the naturally-occurring microbe, but the changes between the two microbes are generally known. In one or more embodiments, the microbe may be or may be derived from a microbe of the class Alphaprotobacteria. In one or more embodiments, the microbe may be or may be derived from a microbe of the phylum Pseudomonadota. In one or more certain embodiments, the microbe may be derived from Pseudomonas stutzeri, Rhodobacter sphaeroides, Gluconacetobacter diazotrophicus, Klebsiella sp., Azospirillum brasilense, Herbaspirillum sp., Burkholderia sp., or Azoarcus sp. G. diazotrophicus is a diazotrophic endophyte that has been investigated for use as a biofertilizer. In one or more preferred embodiments, the diazotrophic microbe is or is derived from wild-type G. diazotrophicus. G. diazotrophicus colonizes the intracellular space of the roots, stems, and leaves of plants. For this reason, it may have potential to efficiently provide nitrogen to crops in co-culture. Endophytes in general, and G diazotrophicus in particular, may more efficiently provide nitrogen to plants as compared to free-living diazotrophs.

Diazotrophs are typically either anaerobic (meaning that they grow in conditions without oxygen), aerobic (meaning that they grow in conditions with abundant oxygen), or microaerobic. In one or more embodiments, the nitrogen-fixing microbe is microaerobic. As used herein, “microaerobic” diazotrophs grow and fix nitrogen in the presence of a low concentration of oxygen. In one or more embodiments, the concentration of oxygen may be at most 21%, at most 19%, at most 17%, at most 15%, at most 12%, at most 10%, at most 9%, at most 8%, 7%, at most 6%, at most 5%, at most 4%, at most 3%, at most 2.5%, at most 2%, at most 1.5%, at most 1%, or at most 0.5% of the atmosphere in which nitrogen fixation occurs. In one or more embodiments, the strain may be grown initially at lower oxygen levels, then grown at an oxygen level approaching atmospheric concentration (e.g., 21%) once the culture density becomes high enough, e.g., when the culture density is sufficient to cause the culture to be limited for oxygen.

The diazotrophic microbes described herein may be genetically modified. In one or more embodiments, the diazotrophic microbe may be genetically modified to increase expression of certain genes, decrease expression of certain genes, knock out certain genes, or introduce genes that the microbe may not naturally have. In one or more embodiments, the diazotrophic microbe may be genetically modified to decrease import of nitrogen-containing compounds into the microbe, to increase secretion of nitrogen-containing compounds, or both. The diazotrophic microbe may be modified to change transport any nitrogen-containing intermediate products in and/or out of the microbe. The diazotrophic microbe may be genetically modified to increase the secretion of nitrogen from the microbe into the external environment. The diazotrophic microbe may be genetically modified to reduce the import of nitrogen-containing compounds from the external environment into the microbe.

The wild-type diazotrophic microbe may include one or more genes encoding an ammonium transporter. Ammonium transporters may function to transport free ammonium back into the cell so that it may be further fixed into ammonia. In one or more embodiments, the diazotrophic microbe includes the ammonia/ammonium transporter gene amtB. amtB gene products typically transport ammonium into the cell and ammonia out of the cell. G. diazotrophicus includes two amtB genes, Gdia_0598 (encoding SEQ ID NO:5) and Gdia_1303 (encoding SEQ ID NO:6). In one or more embodiments, the diazotrophic microbe may include a disruption or deletion of an amtB gene. In one or more embodiments wherein the diazotrophic microbe is derived from G. diazotrophicus, it includes a disruption or deletion of Gdia_0598, a disruption or deletion of Gdia_1303, or both. In one or more certain embodiments, the diazotrophic microbe includes complete deletion of Gdia_1303 and/or Gdia_0598.

While described herein in the context of an exemplary genetic disruption of a nitrogen transporter, disruption, or deletion of other genes within a diazotrophic microbe may improve secretion of nitrogen products. This may further include genes related to nitrogen regulation and genes that result in improved growth during nitrogen fixation, or any gene that competes for resources with nitrogen fixation.

Nitrogen fixation is typically energetically costly to a cell. The amount of nitrogen fixed by a diazotrophic microbe may be limited by the amount of available cellular resources, such as ATP and cofactors. Thus, disruption or deletion of genes that compete for resources may increase nitrogen fixation and secretion of nitrogen-containing compounds, or extracellular polysaccharides that do not improve growth of the bacterium as an endophyte or diazotroph.

While endophytes typically have a symbiotic relationship with co-cultured organisms, microbes can cause an immune response in co-cultured plants. For example, a plant may produce antibiotic compounds in response to detection of certain microbial metabolites. For the co-cultured plant, production of an immune response can be energetically costly and may divert resources away from growth. Thus, to increase growth of co-cultured plants, genes involved in the production of immunogenic compounds within the diazotrophic microbe may be disrupted or deleted.

In one or more embodiments, the microbe may be genetically modified to delete or disrupt a portion of a gene, rather than an entire gene. For example, the Q-linker region of the amt gene and/or the Q-linker region of the NifA gene may be deleted. Deletion of a portion of a gene may result in a modified pattern of expression. This may desirably limit the abundance of a gene product without eliminating it entirely. In particular, this approach may be useful when genetic modification of an essential gene is desired. Exemplary genes that may be targeted for genetic modification include, but are not limited to, Gdia_0213, Gdia_0262, Gdia_0263, Gdia_1645, Gdia_1960, Gdia_2290, Gdia_2292, Gdia_2294, Gdia_2853, Gdia_2854, Gdia_2855, Gdia_2856, Gdia_2914, Gdia_3191, Gdia_3378, Gdia_3522, or Gdia_3523.

Nitrogen may be fixed from gaseous nitrogen (N_{2g}). Nitrogen may be fixed into any form that may be utilized by a plant. Nitrogen-containing compounds that may be used by plants include ammonium (NH₃), urea (CO(NH₂)₂), amino acids, or extracellular peptides or proteins. Diazotrophic microbes may include multiple pathways by which to fix nitrogen. One pathway commonly utilized by microbes to fix nitrogen is the glutamine synthetase/glutamate synthetase pathway (GS-GOGAT).

In one or more embodiments, the microbe may be genetically modified to add one or more genes. Exemplary genetic additions include, but are not limited to, adding repeats of one or more endogenous gene, adding one or more exogenous gene, or both. For example, one can add one or more exogenous genes that increase urea levels in the cell.

In one or more embodiments, the diazotrophic microbe may include one or more polynucleotides encoding a protein involved in nitrogen fixation, such as, for example, a polynucleotide that encodes aminomethyltransferase gcvT (SEQ ID NO:1), NAD+ synthase (SEQ ID NO:2), histidine ammonia-lyase (SEQ ID NO:3), LysR family transcription regulator (SEQ ID NO:4), an ammonium transporter (e.g., SEQ ID NO:5 or SEQ ID NO:6), and/or D-amino acid dehydrogenase (SEQ ID NO:7). Thus, in one or more embodiments, the diazotrophic microbe may include one or more proteins involved in nitrogen fixation, such as, for example, aminomethyltransferase gcvT (SEQ ID NO:1), NAD+ synthase (SEQ ID NO:2), histidine ammonia-lyase (SEQ ID NO:3), LysR family transcription regulator (SEQ ID NO:4), an ammonium transporter (e.g., SEQ ID NO:5 or SEQ ID NO:6), and/or D-amino acid dehydrogenase (SEQ ID NO:7).

The proteins and polynucleotides encoding the proteins identified above are exemplary and are not an exclusive list of proteins encoded by polynucleotides that may be modified to result in desired properties of a diazotrophic microbe. Other genes, encoding other proteins, may be modified and may be identified in any suitable method. For example, a screening method such as a transposon-based screening method may be used to identify additional or alternative genes in a diazotrophic microbe of interest implicated in nitrogen fixation and transport. An example of a transposon-based screening method used to identify genes involved in growth and nitrogen fixation in G. diazotrophicus is described in EXAMPLE 3.

Genetically modified strains typically raise some level of concern as they are thought to increase the risk of genes being transferred to different organisms. In one or more embodiments, a gene modification strategy is used that does not introduce undesirable exogenous genes (e.g., antibiotic resistance genes or other selection-related genes) to the modified organism. As used herein, “undesirable exogenous genes” are any genes that do not naturally occur within an organism and that are considered undesirable in a genetically modified organism. Undesirable exogenous genes may include markers commonly used to detect genetic modification of an organism, such as an antibiotic resistance gene, a colorimetric marker such as the lacZ system, or a fluorescent protein. Diazotrophic microbes that do not include any exogenous genes may present fewer risks to the environment than diazotrophic microbes that include undesirable exogenous genes, particularly antibiotic resistance genes. Genetically modified strains often raise public concern, as they are thought to increase the risk of genes being transferred to other organisms within the environment. The gene disruptions and deletions described herein may be “clean deletions,” meaning that no portion of the deleted gene is left behind, and expression of the gene is not removed by inserting a different gene at a given locus. In one or more embodiments the genetic modification of a diazotrophic microbe reduces the growth rate of the bacteria as compared to a wild-type microbe. When a genetically modified microbe shows lower fitness than a wild-type strain, the wild-type strain may outcompete the modified strain, resulting an eventual decline in numbers. Thus, genetically modified strains may pose a minimal risk of invasive behavior if used outside of a laboratory setting.

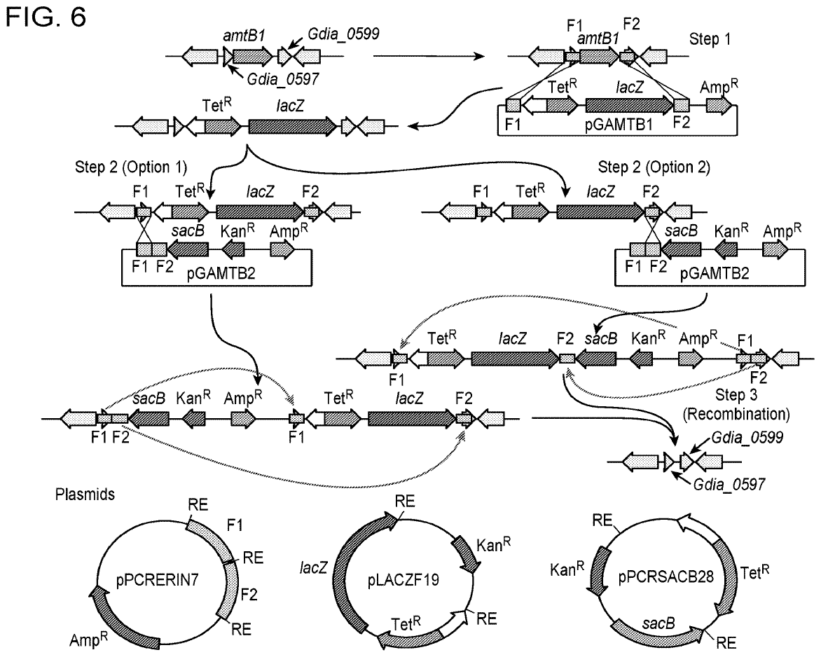

FIG. 6 provides an exemplary strategy for manipulating the genome of G. diazotrophicus so that the resultant modified microbe does not retain any selection-related genes. Using two steps that first replace an amtB gene with a visual marker (lacZ) through a double homologous recombination event and then introduce the conditionally toxic sac gene through a single homologous recombination, a strain containing multiple flanking regions with a strong potential for deleting the visible and selectable markers was constructed. The desired deletion was identified by growing in GADN medium two times without antibiotic pressure, and then transferring to medium supplemented with 20% sucrose. High levels of sucrose were required to achieve a sacB-dependent growth deficiency in G. diazotrophicus. After selection, deletions were identified by plating on GADN plates containing X-Gal to identify colonies that no longer carried the lacZ gene and linked antibiotic markers and then confirmed by PCR. Applying this approach, clean gene deletions were constructed for both amtB1 and amtB2 (Gdia_0598 and Gdia_1303 respectively). These two amtB genes were deleted sequentially and were confirmed by PCR with primers external to the manipulated regions and then by antibiotic challenge to confirm loss of the antibiotic markers in the final construct. For the completed constructs, the entire gene was removed from these strains, confirming that the desired modification was successful in each case.

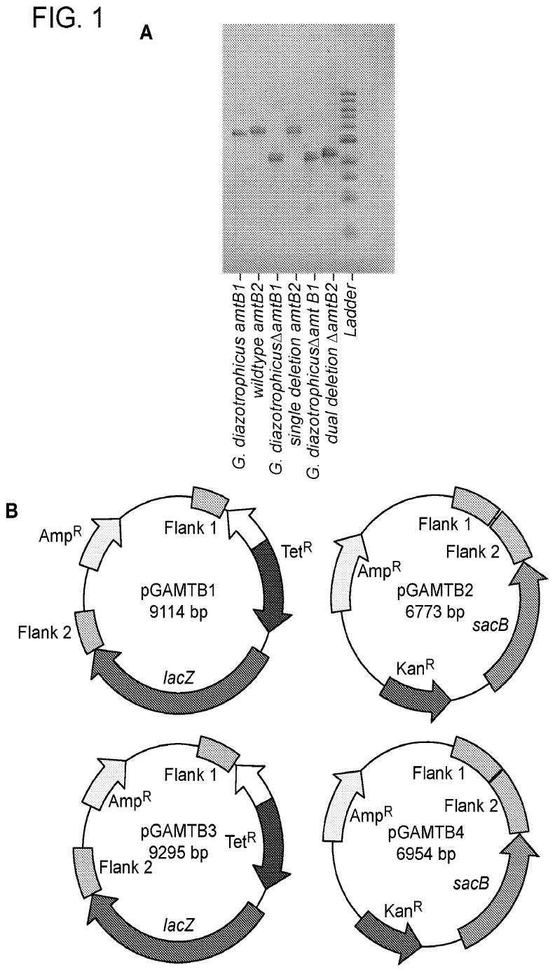

In addition to the ability to remove genes in their entirety with no remaining antibiotic markers, the genome editing method described here also enables additional and more strategic modifications or even the introduction of exogenous genes, again without leaving an antibiotic marker behind. This is because manipulations made to the plasmid in the single homologous second recombination step of the genome editing method (step 2 in FIG. 1) are incorporated into the genome following the internal recombination under selective pressure based on sucrose in combination with sacB. This was demonstrated by first removing a small segment of the nifA gene from G. diazotrophicus using the double homologous approach in step one of the gene editing protocol. The plasmid containing the flanking regions was further modified to construct the desired genome modification, which here was removal of a series of codons encoding the Q-linker region of nifA. Once confirmed, the sacB and kanamycin cassette were again added to the plasmid outside of the segment containing the desired modifications and flanking regions to the manipulated Q-linker region. In this manner, internal recombination during the final step of the gene editing method that removes the lacZ gene and tetracycline marker also introduces the newly manipulated region back into the genome in a seamless manner. This was confirmed both by itself (GABB027) and in combination with the dual amtB deletion strain (GABB040) by PCR of the final genome segment. Following confirmation by PCR, the segment of the genome containing the Q-linker was sequenced by Sanger sequencing to confirm removal of the specific codons coding for the Q-linker amino acids (DRENLLHDSGLAQPAAPVAD (SEQ ID NO:28) to DRENLAPVAD (SEQ ID NO:29).

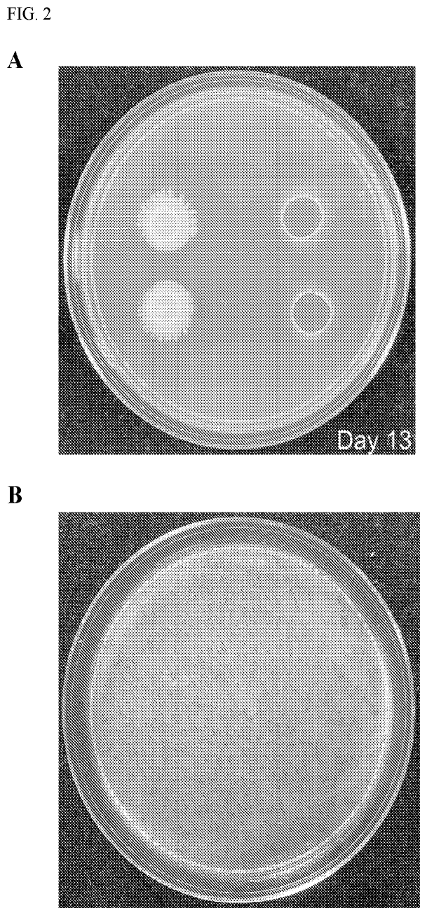

In one or more embodiments, application of the diazotrophic microbes described herein may increase plant growth as compared to a plant treated with a wild-type diazotrophic microbe. These differences are typically attributed to the genetic modifications and their downstream effects. Increased plant growth may be due to the genetically modified diazotrophic microbe providing higher nitrogen concentrations outside the cell compared to a wild-type diazotrophic microbe. An example of a genetically modified diazotrophic microbe providing increased levels of extracellular fixed nitrogen is described in EXAMPLE 1 and shown in FIG. 2.

In order to test for elevated nitrogen production under diazotrophic conditions, G. diazotrophicus was grown micro-aerobically. G. diazotrophicus requires micro-aerobic conditions to grow diazotrophically on solid medium as the nitrogenase enzyme is sensitive to oxygen. When grown on solid agar plates, diazotrophic growth required a stream of 5% O₂ in the headspace. Different algal strains were tested to confirm the ability to grow under this low oxygen environment. Chlorella sorokiniana grew well alone under this low oxygen concentration when provided with alternating light and dark cycles (8 hours each) on plates provided with supplemented nitrogen in the form of nitrate.

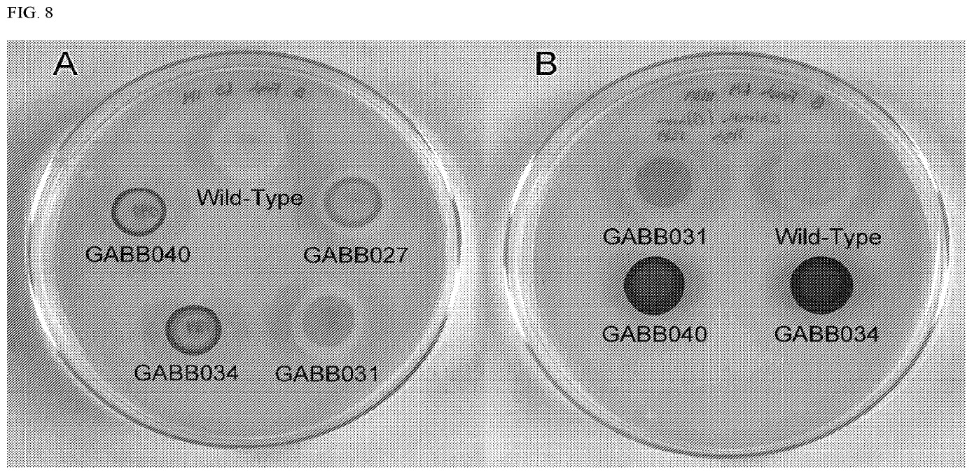

Since C. sorokiniana requires nitrogen to grow and produces green photosynthetic pigments, it can be used as a biosensor for available nitrogen, and can be distinguished from G. diazotrophicus in combination on plates (FIG. 8). The wild-type strain generates minimal extracellular nitrogen, resulting in a slow bleaching of the algal cells over the course of a week. The single amtB1 deletion (GABB031) and the Q-linker deletion (GABB027) result in minimal growth of C. sorokiniana, indicating slight improvement in external nitrogen release. The dual amtB deletion strain (GABB034) and the combined Q-linker with the dual amtB deletion (GABB040) showed a significant affect in the phenotype with C. sorokiniana. This change in phenotype indicates a significant increase in external nitrogen since the growth medium in the plates is devoid of fixed nitrogen. In addition to an increase in extracellular nitrogen, these strains also produce a lower amount of extracellular polysaccharides, manifesting as a less goopy phenotype that protrudes outside of the initial spotted cells (FIG. 8).

Initial growth of G. diazotrophicus under diazotrophic conditions requires a micro-aerobic atmosphere to maintain the culture in planktonic form. When G. diazotrophicus is cultured in a turbidostat reactor that allowed us to maintain a micro-aerobic atmosphere over the culture during the initial stage of growth, cultures achieved a cell density of approximately 0.1 to 0.2 OD₆₀₀. Denser diazotrophic growth was not possible under these conditions unless the percent oxygen concentration of the atmosphere was incrementally increased. Once the atmosphere was shifted to a standard atmosphere with a slow flow rate, cell densities of approximately 0.4 to 0.5 OD₆₀₀ were possible in the turbidostat, with the majority of the culture remaining in a planktonic phase of growth. These findings differ from prior reports that have characterized nitrogen fixation in G. diazotrophicus (Cavalcante et al., 1988. Plant and Soil 108:23-31; Fisher et al., 2005. Biochimica Et Biophysica Acta-Proteins and Proteomics 1750:154-165; Stephan et al., 1991. FEMS Microbiol Lett 77:67-72), and indicate that nitrogen fixation (and as a result, culture density) are oxygen limited at higher density, but require micro-aerobic conditions at low density to transition from non-diazotrophic growth conditions. In addition to requiring elevated densities, the cells also grew better if they remained in a planktonic phase of growth, as they were prone to form aggregates if growth was slow through this phase. Reversing the culture back to a planktonic growth phase was difficult under diazotrophic growth once the culture had begun to aggregate.

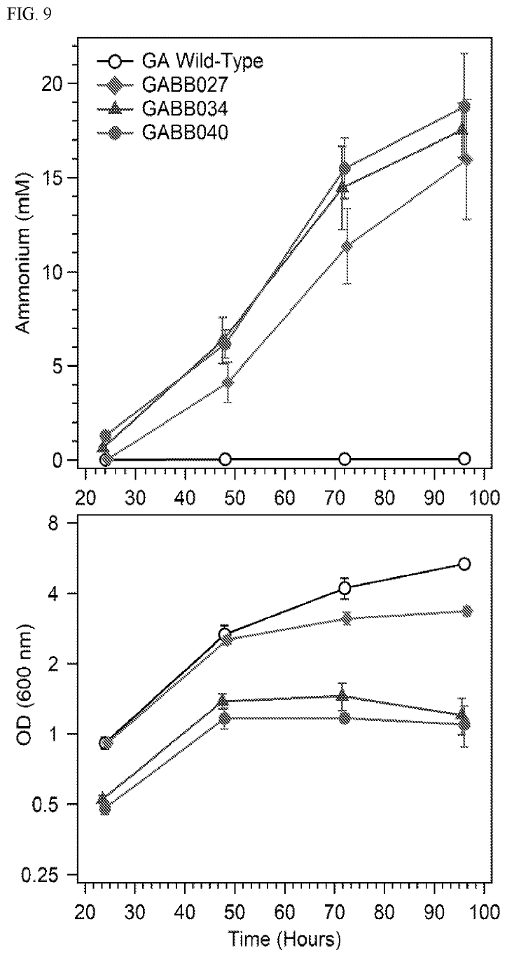

To overcome the issues associated with transitioning cultures from micro-aerobic conditions in turbidostats to fully aerobic growth and also maintain the cells in a planktonic state of growth, a protocol was developed to transition the cells to an OD₆₀₀ of approximately 0.5 by transferring a small aliquot of cells from GADN medium (in planktonic growth) to a medium containing a limiting amount (1.25 mM) of asparagine, which can serve as a limited nitrogen source. Once cells achieved this 0.5 OD₆₀₀ (in the absence of any aggregates), the cells were able to grow diazotrophically under aerobic conditions, in a manner similar to the conditions used to generate extracellular ammonium from Azotobacter vinelandii. Strains GABB027 (ΔQ-linker), GABB034 (ΔamtB1, ΔamtB2) and GABB040 (ΔQ-linker, ΔamtB1, ΔamtB2) all achieved high levels of ammonium (16-19 mM) after four days of growth (FIG. 9), while wild-type G. diazotrophicus remained below 0.1 mM ammonium throughout the entire 4 days. These quantities well exceed the potential nitrogen that could be derived from the 1.25 mM of asparagine used to support the initial growth of the cells while transitioning to aerobic diazotrophic growth. Cell density for wild-type G. diazotrophicus achieved an OD₆₀₀ of 5.3 after four days, while GABB027 achieved 3.3, and GABB034 and GABB040 respectively reached only 1.4 and 1.1 OD₆₀₀. These results correlate well with the algal co-culture plate experiments, and revealed that levels of ammonium achieved for GABB027, GABB034 and GABB040 were considerably higher than what could be achieved with wild-type, with minor growth defects found for increasing complexity of the gene deletions, manifesting as a lower final cell density achieved.

Thus, in one or more embodiments, the genetically modified diazotrophic microbe may increase extracellular nitrogen by at least two-fold, at least 10-fold, at least 100-fold, at least 500-fold, at least 1000-fold, at least 2000-fold, or at least 3000-fold as compared to a wild-type diazotrophic microbe. In one or more embodiments, the genetically modified diazotrophic microbe may increase extracellular nitrogen by at most 10,000-fold, at most 5000-fold, at most 4000-fold, or at most 3000-fold as compared to a wild-type diazotrophic microbe.

In one or more embodiments, use of the genetically modified diazotrophic microbe may yield an extracellular nitrogen concentration greater than the extracellular nitrogen concentration produced by a wild-type strain of the diazotrophic microbe, which is typically 10 micromolar (μM). Thus, in one or more embodiments, the genetically modified diazotrophic microbe may yield an extracellular nitrogen concentration of, for example, at least 25 μM, at least 50 μM, at least 100 μM, at least 500 μM, at least 1 millimolar (mM), at least 5 mM 8 millimolar, at least 10 mM, at least 12 mM, at least 15 mM, or at least 20 mM. In one or more embodiments, use of the genetically modified diazotrophic microbe may yield an extracellular nitrogen concentration of at most 35 mM, at most 30 mM, at most 28 mM, at most 26 mM, at most 24 mM, or at most 22 mM.

The purpose of this work was to develop an elevated nitrogen producing endophyte diazotrophic strain for potential future application as a biofertilizer, and compare nitrogen levels obtained to other strains. The endophyte G. diazotrophicus was engineered to produce elevated levels of extracellular nitrogen as ammonium. Genetic modification may raise concern due to the potential to introduce foreign genes into the environment, often in the form of antibiotic resistance used for selection during strain construction. Although the strains constructed here were genetically altered, this disclosure describes an approach that generates clean gene deletions (FIG. 6). Clean deletions do not leave any antibiotic resistant markers behind in the final strain. The strain simply has genes removed that were originally obtained through horizontal gene transfer or evolution. The removal of genes often results in a disadvantage for the bacteria. The wild-type strain should outcompete this modified strain, resulting in an eventual decline in numbers for manipulated strains. Hence, there should be minimal concern for this strain if applied as a biofertilizer.

The selection of Chlorella sorokiniana as an exemplary surrogate for higher land plant studies capitalized on the rapid growth rate and ability to test the strain on solid medium under micro-aerobic conditions within a sealed chamber. C. sorokiniana is unable to fix nitrogen, and is thus dependent on G. diazotrophicus for nitrogen, serving as a biosensor for extracellular nitrogen. Amounts of ammonium accumulated in medium as a result of ammonium transport abolishment tend to be very low, in the μM range in liquid culture for other strains, so the use of a biosensor is a convenient alternative for measuring these low levels of release by growing the two strains in close proximity. Elevated levels of algal growth for the Q-linker deletion strain GABB027 indicates that disruption of the NifA Q-linker alone in G. diazotrophicus is sufficient for a minimal increase in extracellular ammonium under micro-aerobic conditions. When grown to high density under aerobic growth, the disruption of the NifA Q-linker was sufficient to yield high levels of ammonium, similar to what was obtained with more extensively manipulated constructs such as GABB040. Actual concentrations of ammonium that accumulated in G. diazotrophicus for the dual amtB deletion strain GABB034 achieved levels of ammonium surpassing 17 mM under optimal condition. This is significantly higher than what has been reported for amtB disruptions in other strains (15, 19, 41), where levels of ammonium only achieved low μM concentrations (Barney et al., 2015. Appl Environ Microbiol 81:4316-4328; Castorph et al., 1984. Arch Microbiol 139:245-247; Zhang et al., 2012. Res Microbiol 163:332-339). The combination strain of the dual amtB gene deletion with the Q-linker disruption achieved a similar ammonium concentration of 18 mM. While G. diazotrophicus requires either a phase of growth under micro-aerobic conditions to achieve sufficient cell density in a planktonic form or a minimal nitrogen source during the transition to diazotrophic growth, once that density is achieved, the strain can be grown to higher density under a standard aerobic atmosphere (FIG. 9), and further indicate that the cells actually require elevated oxygen to grow to high density as a diazotroph. For this reason, G. diazotrophicus may therefore be characterized as conditionally micro-aerobic in contrast to micro-aerobic for diazotrophic growth, as it is generally classified. The development of this approach for testing diazotrophic growth of G. diazotrophicus without the need for a micro-aerobic atmosphere, should be useful for future assays of this microbe, as it is highly reproducible and easy to scale (FIG. 9).

In use, it is thought that G. diazotrophicus enters into the plant through cracks at lateral root emergence sites. Thus, G. diazotrophicus could be applied to seeds just prior to planting, allowing it to enter the plant during early stages of growth and live in the plant throughout its lifespan. G. diazotrophicus could also be applied to the soil during early plant growth, allowing it to enter the plant at this time. Another way endophytes can be introduced to the plants is through the stomata in the leaves of the plant. This would allow G. diazotrophicus to be applied to the plant at later stages, potentially by application using precision farm equipment. The results provided herein indicate significant improvements in extracellular ammonium production for G. diazotrophicus that can be monitored using either a microalga as a biosensor on solid plates, or by direct methods to quantify ammonium, even when grown under a standard atmosphere.

The characteristics of G. diazotrophicus as a growth promoting endophyte and the application of genetic approaches here for its manipulation provide multiple opportunities to further tailor this strain to improve specific functions. The deletion of the two ammonium transporting genes along with removal of the Q-linker from NifA increases the amount of nitrogen released outside of the cell. It also demonstrates that the amount of ammonium that can be obtained from G. diazotrophicus using only amtB deletions is far higher than what has been reported for other strains.

Thus, in another aspect, the present disclosure describes a composition including a genetically modified endophytic diazotrophic microbe. The composition may include additional nutrients, microbes, or components that may be beneficial to a plant. The compositions described herein may be applied to an organism of interest, such as a plant, in any suitable fashion.

The compositions described herein may be formulated with any agriculturally suitable carrier. As used herein, “carrier” includes solvent, dispersion medium vehicle, coating, diluent, suspension, colloid, and the like. The compositions described herein may be applied as a liquid, such as a spray, through an irrigation system, as a lyophilized powder, in pellets, or in any other suitable form.

As used herein, “agriculturally acceptable” refers to a material that is not biologically or otherwise undesirable, e.g., the material may be applied across a small or large plot of land without undesired negative ecological impact. The compositions described herein may be administered to any suitable location, including to leaves, seeds, soil, roots, root emergence sites, stem, or meristem. In one or more embodiments, the composition may be applied to a naturally-occurring crack or to an introduced break in the plant wall, such as to a crack at a lateral root emergence site or to a cut introduced to a leaf. Some endophytes are known to enter co-cultured plants through the stomata. Thus, the composition may be applied directly to one or more leaves. The composition may be applied at any point during development, or it may be added to a growth media such as soil prior to introduction of a plant.

The compositions described herein may be applied to a plant with any suitable frequency. For example, the composition may be applied once per day, twice per week, once per week, twice per month, once per month, or once per year. The composition may be reapplied based on a predetermined metric, such as the concentration of nitrogen in the soil, or the rate of plant growth.

### Methods

In another aspect, the present disclosure describes a method of increasing growth of an organism in co-culture with a genetically modified endophytic diazotrophic microbe. Generally, this method includes co-culturing an organism with a genetically modified endophytic diazotrophic microbe. The genetically modified endophytic diazotrophic microbe may be consistent with any described herein. In one or more embodiments, the genetically modified diazotrophic microbe is within the organism in co-culture. For example, if the organism in co-culture is a flowering plant, the microbe may be within the leaves, roots, stem, and/or flowers of the plant.

In one or more embodiments, the organism in co-culture is not a diazotroph. The organism may be a photosynthetic organism, such as a plant or an alga. The plant may be a vascular plant, such as a legume, tree, or grass. The organism may be a biofuel feed crop, such as corn, soy, algae, sugarcane, sugar beet, sorghum, or canola.

In one or more embodiments, increasing the growth of the organism includes increasing the growth rate of the organism. In one or more embodiments, increasing the growth of the organism includes increasing the cell density of the organism. In one or more embodiments, increasing the growth of the organism includes increasing the crop yield of the organism. In one or more embodiments, increasing the growth of the organism may be a result of increased nitrogen availability.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXAMPLES

The present invention is illustrated by the following examples. It is to be understood that the particular examples, materials, amounts, and procedures are to be interpreted broadly in accordance with the scope and spirit of the invention as set forth herein.

TABLE 1
Reagents Used in the Examples

| Reagent | Source/Cat. No. |
|---|---|
| GAD medium | Schwister et al., Appl Indust Microbiol, |
| | “Gluconacetobacter diazotrophicus Gene |
| | Fitness during Diazotrophic Growth” Nov. 14, |
| | 2022. |
| GADN medium | Schwister et al., Appl Indust Microbiol, |
| | “Gluconacetobacter diazotrophicus Gene |
| | Fitness during Diazotrophic Growth” Nov. 14, |
| | 2022. |
| Lysogeny broth | Thermo Fisher Scientific |
| Escherichia coli JM109 | New England Biolabs, Inc., Ipswich, MA |
| Primers | Integrated DNA Technologies, Inc., |
| | Coralville, IA |
| Tetracycline | Thermo Fisher Scientific or Sigma Aldrich |
| Kanamycin | Thermo Fisher Scientific or Sigma Aldrich |
| 5-bromo-4-chloro-3-indolyl-β-D- | Thermo Fisher Scientific or Sigma Aldrich |
| galactopyranoside (X-gal) | |
| Burks medium | Schwister et al., Appl Indust Microbiol, |
| | “Gluconacetobacter diazotrophicus Gene |
| | Fitness during Diazotrophic Growth” Nov. 14, |
| | 2022. |
| Sodium chloride | Thermo Fisher Scientific or Sigma Aldrich |
| Sodium sulfate | Thermo Fisher Scientific or Sigma Aldrich |
| Sodium citrate | Thermo Fisher Scientific or Sigma Aldrich |
| Plate Biology Grade Agarose | Thermo Fisher Scientific or Sigma Aldrich |
| 2,6-di-aminopimelic acid | Thermo Fisher Scientific or Sigma Aldrich |
| ZR Fungal/Bacterial DNA MiniPrep Kit | Zymo Research |
| GenePulser | BioRad Laboratories, Inc., Hercules, CA |
| Acoustic DNA shearing device | Covaris, LLC, Woburn, MA |
| G. diazotrophicus PA1 5 stock | ATCC, 49037 |
| C. sorokiniana stock | UTEX Culture Collection of Algae |
| E. coli WM3064 stock | ATCC |

### Example 1

In this Example, amtB1 and amtB2 were genetically removed from wild-type G. diazotrophicus to prepare the ΔamtA1 ΔamtB2 G. diazotrophicus strain. All reagents used in this Example were obtained, for example, from Sigma-Aldrich, Thermo Fisher Scientific, Integrated DNA Technologies, and New England Biolabs.

Deletion of amtB1 and amtB2

Plasmids pGAMTB1, pGAMTB2, pGAMTB3, and pGAMTB4 (shown in FIG. 1B) were prepared following standard molecular biology approaches including amplification, modification and insertion of and then removal of selective markers. Each plasmid was expressed and purified using standard molecular biology techniques.

For plasmid transformations, G. diazotrophicus PA1 5 (ATCC 49037) was grown aerobically at 30° C. on GAD basal medium. GAD basal medium was adapted from DYGS basal medium and included 2 g/L glucose, 2 g/L yeast extract, 1.5 g/L tryptone, 0.5 g/L magnesium sulfate, and 1.5 g/L glutamic acid. GAD medium was adjusted to pH 6.2 with sodium hydroxide. GAD media used to isolate G. diazotrophicus with transposon inserts was supplemented with 100 mg/L tetracycline.

Transformants were screened using lacZ/X-gal colorimetric selection. A representative plate, wherein double transformants are white, is shown in FIG. 2B.

Using both single and double homologous recombination, amtB1 (Gdia_0598) and amtB2 (Gdia_1303) were deleted from a modified G. diazotrophicus stock. Each gene was deleted sequentially, as confirmed by a reduction in PCR product size as shown in FIG. 1A. The strain of G. diazotrophicus with both amtB1 and amtB2 deleted is referred to as ΔamtB1B2 G. diazotrophicus.

The entire gene was removed from the strain, as illustrated by a smaller PCR product obtained from primers that flank the location of the modification, confirming that the desired modification was successful (FIG. 1A).

### Example 2

In this Example, ΔamtB1B2 G. diazotrophicus was co-cultured with C. sorokiniana to determine whether ΔamtB1B2 G. diazotrophicus was able to provide usable nitrogen to an organism in co-culture.

G. diazotrophicus was first grown independently on plates of modified B medium. Modified B medium was adapted from Burk's medium and included 50 mM sodium citrate, 20 g/L sucrose, 0.2 g/L magnesium sulfate heptahydrate, 0.09 g/L calcium chloride dihydrate, 0.025 g/L sodium molybdate dihydrate, and 0.05 g/L iron sulfate heptahydrate. Buffer components were combined with 2 mL of 100× phosphate buffer (20 g potassium dihydrogen phosphate and 80 g of dipotassium hydrogen phosphate in 1 L of water), and the total volume was raised to 1 L and pH adjusted to pH 6.2 with NaOH. Plates were prepared using agarose.

C. sorokiniana UTEX 1602 from was grown photoautotrophically and independently on a basal freshwater medium using fluorescent or LED light banks and a standard atmosphere to fix carbon dioxide. C. sorokiniana lacks the ability to fix nitrogen from the atmosphere.

The co-culture was grown micro-aerobically in an atmosphere including 5% oxygen. These conditions were necessary to enable nitrogen fixation by G. diazotrophicus, as the nitrogenase is sensitive to atmospheric oxygen. C. sorokiniana was found to grow well in an atmosphere of 5% oxygen as well. G. diazotrophicus and C. sorokiniana were co-cultured on LB plates lacking a nitrogen source in a microaerobic atmosphere (2.5% oxygen).

Growing G. diazotrophicus with C. sorokiniana helped visualize the level of nitrogen production in the mutant strain of G. diazotrophicus. C. sorokiniana grown with wild-type G. diazotrophicus was a pale yellow color (FIG. 2A). C. sorokiniana grown with ΔamtB1B2 G. diazotrophicus was a bright green color (FIG. 2A). The bright green color indicates C. sorokiniana growth. As there was no nitrogen in the media, this indicates that ΔamtB1B2 G. diazotrophicus provided a source of usable nitrogen to the C. sorokiniana.

From this Example, it was learned that ΔamtB1B2 G. diazotrophicus was able to provide usable nitrogen to another organism in co-culture.

### Example 3

In this Example, a library screen was completed to identify genes involved in G. diazotrophicus fitness and nitrogen fixation during diazotrophic growth.

### Library Growth

Two conditions were selected for comparisons of diazotrophic growth. The first condition utilized a modified Burk's medium with sucrose as the primary carbon source, supplemented with ammonium sulfate. This culture was grown under a standard atmosphere and grew to approximately 7 generations. The second condition was grown with the same medium in the absence of a supplemented nitrogen source to encourage diazotrophic growth, and was grown in a closed reactor system with a low oxygen (2.5% by volume) atmosphere, as it was not possible to initiate this diazotrophic growth under a standard atmosphere. Under these conditions, the culture grew for approximately 4 generations before entering stationary phase. Both conditions were grown with citrate as a buffer system to maintain the lower pH preferred by G. diazotrophicus.

### Library Statistics

The Mariner transposon integrates at TA dinucleotides. The complete genome of G. diazotrophicus has 58,303 TA sites. Of these sites, 57,574 are within the chromosome (CP001189.1) and 729 are within the plasmid (CP001190.1). Joint Genome Institute (JGI) assembly was used as reference library and both assemblies were used for annotation. A total of 8,979 of all TA sites were found to be non-unique while 3,773 were non-permissive. In some cases, TA sites were both homologous and non-permissive. Due to the indiscriminate and restrictive features of these regions, we removed a total of 8,668 TA sites (14.87%), 8,641 from the chromosome and 27 from the plasmid. In addition, we filtered out any TA sites found in the first five percent and last ten percent of genes based on concerns that some genes can tolerate insertions in these regions and still retain gene function. After this filtering step, 26,349 TA sites were removed (44.43%), leaving a total of 31,954 TA sites remaining (58.81%). The remaining TA sites covered 3,286 genes out of 3,501 (93.9%). While not 100% complete, this library yielded acceptable coverage for a transposon-created library, and provided sufficient data to probe 3,286 of the 3,501 genes listed in the JGI assembly and annotation.

### Major Nif Cluster Genes

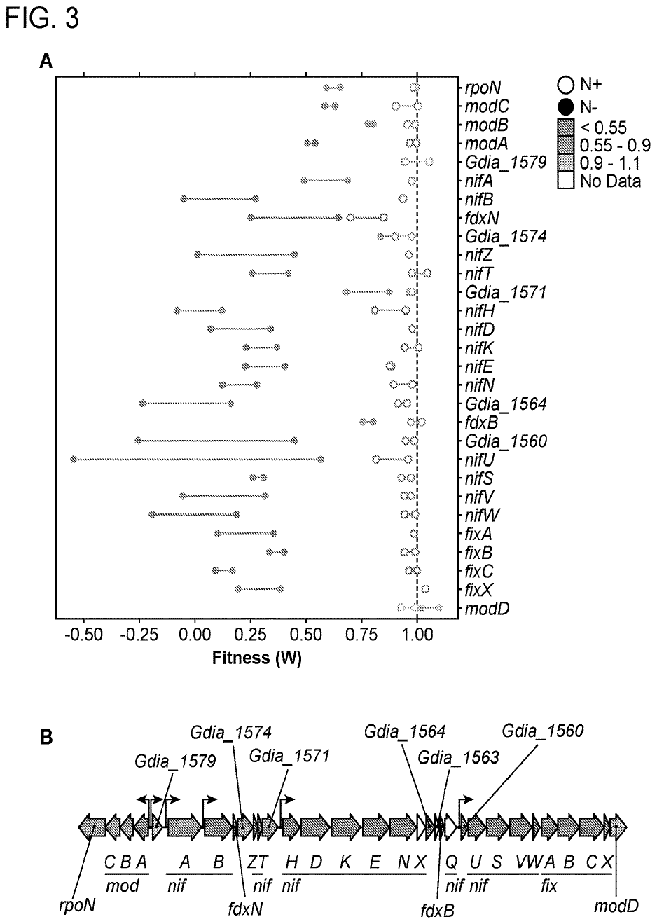

The initial intent of this project was to explore the fitness values of genes related to molybdenum-dependent nitrogen fixation in G. diazotrophicus when grown in the presence of ammonium and under nitrogen fixation conditions. Fitness values for single genes provide a measure of the importance that a gene plays in a given process. Unlike model diazotrophs such as Azotobacter vinelandii, Pseudomonas stutzeri and Rhodobacter sphaeroides, the major genes associated with molybdenum-dependent nitrogen fixation in G. diazotrophicus are arranged in one primary cluster (FIG. 3B). The fitness values of this major gene cluster of nif and nif-related genes are presented in FIG. 3A. The genes were assigned to one of several categories based upon their fitness value. The categories were as follows: a large growth defect (purple), moderate defect (blue), and no or minimal defect (green). Genes that were unable to be analyzed based on the lack of sufficient data or the absence of suitable TA sites are shown in white. Under diazotrophic growth, the structural genes encoding nitrogenase (nifH and nifDK) were found to have large fitness losses (FIG. 3). These genes are associated with the α- and β-subunits of the MoFe protein (nifDK) and the Fe (nifH) protein that provides the electrons to the MoFe protein. Similar to nifH and nifDK, the genes nifB, nifZ, nifT, nifEN, nifS, nifV, nifW, Gdia_1564 and modA all showed large differential fitness values under diazotrophic growth. Many of these genes have assigned function in MoFe cofactor biosynthesis. Additional genes resulted in fitness losses, but higher variability in the results, including nifU, fdxN and Gdia_1560. The four genes of the fixABCX cluster that are part of this larger cluster also showed a pronounced fitness defect under diazotrophic conditions. The rpoN, modC, modB and fdxB genes showed slightly lower gene defects. Genes Gdia_1579 and modD showed minimal differences.

### Nitrogen Assimilation and Regulation Genes

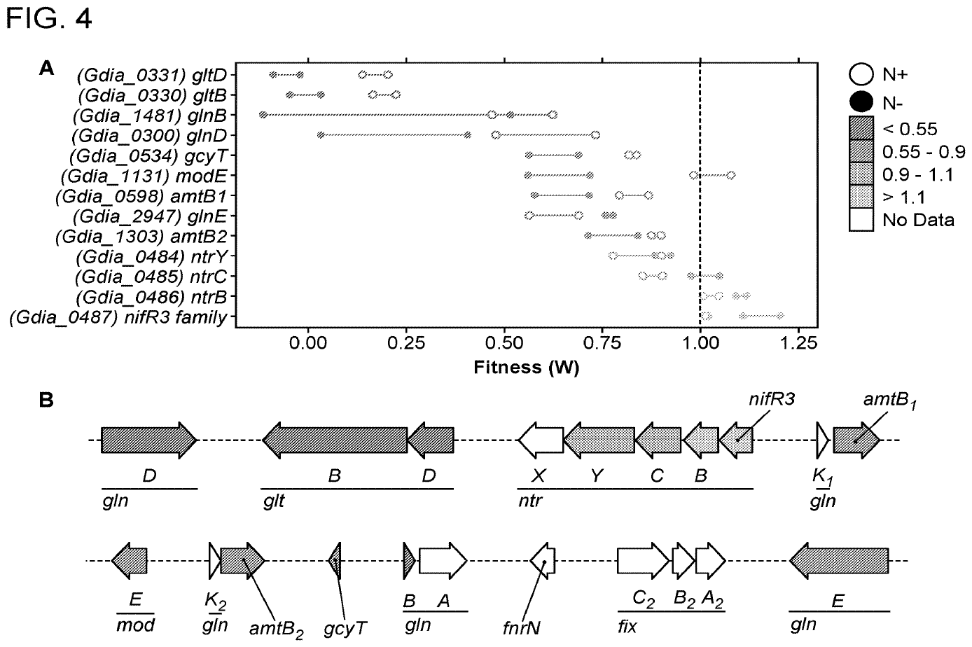

Beyond the genes within the major nif cluster, are the proteins associated with nitrogen assimilation and regulation. These genes range in function from ammonium uptake into the cell, to activation of nif gene transcription, and many are not included in the major nif cluster. Rather, these genes are scattered throughout the genome, some organized in operons and some independently transcribed. The fitness values of these genes associated with nitrogen assimilation and regulation are presented in FIG. 4. Like the major nif cluster, these genes also fell into fitness categories consistent with them having a role in growth under diazotrophic conditions.

G. diazotrophicus contains limited functional redundancy in genes related to nitrogen fixation. However, this microbe does display some genetic homologs with redundancy in genes related to nitrogen regulation. G. diazotrophicus contains two homologs of the ammonium transporter amtB, termed amtB1 (Gdia_0598) and amtB2 (Gdia_1303), which bring ammonia into the cell. While in separate genomic regions, both genes displayed moderate fitness defects under growth with or without nitrogen included (FIG. 4), suggesting a general fitness independent of diazotrophic growth associated with these genes, though this defect increased under diazotrophic growth.

A number of the nitrogen-associated gene disruptions showed strong fitness defects, regardless of whether these were provided ammonium, or grown diazotrophically. The four genes gltD (Gdia_0331), gltB (Gdia_0330), glnB (Gdia_1481), and glnD (Gdia_0300) all showed significant defects that increased under conditions of diazotrophic growth. The glnB and glnD genes showed higher variability. In contrast, glnE (Gdia_2987) showed a lesser growth defect when provided with ammonium, but that defect was slightly minimized under diazotrophic growth. The nifR3 family (Gdia_0487) and ntrBCY genes (Gdia_0484-0486) all showed minimal or no defects when provided ammonium, but each had minor improvements under diazotrophic growth, similar to glnE. A number of other genes of interest (in particular, smaller genes) that might be associated with nitrogen fixation were unable to be analyzed through this approach.

### Additional Genes Exhibiting Diazotrophic Growth Defects

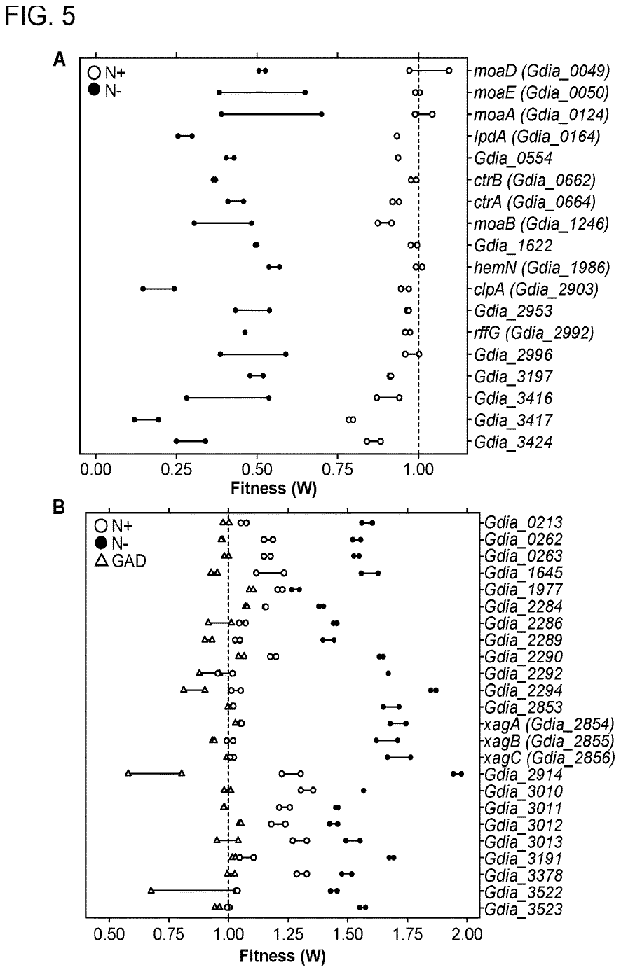

In addition to the genes of the major nif cluster, additional genes that contribute to fitness under diazotrophic conditions were identified. FIG. 5A shows a selection of genes which exhibited significant growth defects under diazotrophic conditions when these genes were disrupted by transposon insertions. Genes with high variability or substantial fitness losses in medium containing supplemented ammonium were omitted from FIG. 5A. Results were ordered in a manner to cluster genes that were in close proximity to one another within the genome. Genes found to show significant defects under diazotrophic growth included clpA (Gdia_2903), a YgpP/YjpQ family permease (Gdia_3416 and Gdia_3417), ctrAB (Gdia_0660, 0662 and 0664), and genes coding the molybdenum cofactor biosynthesis proteins moaBDE (Gdia_1246, 0049 and 0050, respectively), and several hypothetical or conserved hypothetical genes.

### Gene Disruptions Resulting in Fitness Improvements

These results revealed a number of gene disruptions that improved fitness of G. diazotrophicus under diazotrophic growth, or with ammonium provided, or in both cases (FIG. 5B). In FIG. 5B includes a selection of genes with considerable fitness increases, and minimal variability between replicates. The genes shown in FIG. 5B are arranged in order as they appear in the genome, so that clusters of genes, such as Gdia_0262 and Gdia_0263, Gdia_2284 through Gdia_2294, xagABC, and others can be compared. Many of these genes resulted in quite dramatic improvement in growth, especially when grown diazotrophically under microaerobic conditions, similar to the minor differences seen for ntrB in FIG. 4A.

From this Example, additional genes that may be deleted or disrupted to improve growth and/or nitrogen fixation in G. diazotrophicus.

### Example 4

### Bacterial Strains and Growth Conditions

Gluconacetobacter diazotrophicus PA1 5 (ATCC 49037) was grown aerobically at 30° C. on GADN medium (All per liter; 2 g glucose, 2 g yeast extract, 1.5 g tryptone, 0.5 g MgSO₄·7H₂O, 1.5 g sodium glutamate, 200 mg ammonium sulfate and 200 mg asparagine, adjusted to pH 6.2 with HCl). For diazotrophic growth plate experiments, a modified Burk's (B) medium was utilized that contained 50 mM sodium citrate as buffer, 20 g sucrose, 200 mg MgSO₄·7H₂O, 90 mg CaCl₂·2H₂O, 0.25 mg Na₂MoO₄·2H₂O, 5 mg FeSO₄·7H₂O and 2 mL of a phosphate buffer composed of 20 g KH₂PO₄ and 80 g K₂HPO₄ in 1 L dH₂O; adjusted to pH 6.2 with NaOH or HCl. GADN media used to isolate G. diazotrophicus following plasmid manipulations was supplemented with 100 mg L⁻¹ tetracycline and/or 200 mg L⁻¹ kanamycin. When grown together with algae, this medium was further supplemented with 1 mL per liter of micronutrient solution as previously described (Barney et al., 2015. Appl Environ Microbiol 81:4316-4328; Knutson et al., 2018. Algal Res 35:301-308) and substituted with plant growth grade agar.

G. diazotrophicus Strain Construction

Plasmids were constructed within Escherichia coli JM109 (New England Biolabs, Inc., Ipswich, MA) as previously described (Barney et al., 2015. Appl Environ Microbiol 81:4316-4328; Eberhart et al., 2016. J Appl Microbiol 120:1595-1604). Strains constructed for this study are listed in Table 2. The plasmids for the construction of G. diazotrophicus strains are described in Table 3. Primers required for construction of the strains are listed in Table 4. Plasmids were transformed into G. diazotrophicus through electroporation as described previously (Teixeira et al., 1999. FEMS Microbiol Lett 176:301-309; Schwab et al., 2016. Arch Microbiol 198:445-458). DNA transfer was accomplished by growing G. diazotrophicus in liquid GADN medium until it had reached an OD₆₀₀ of approximately 1.0, then 1 mL of cells were spun down at 7000×g and washed two times with 10% glycerol in distilled water and resuspended in 100 μL. These cells were electroporated in 0.1 cm gap electroporation cuvettes using a gene pulser (BioRad Laboratories, Inc., Hercules, CA) set at 600Ω resistance, 25 μF capacitance, 12.5 kV/cm and a pulse length of ˜12 ms. After electroporation, the cells were transferred to 50 mL of fresh GADN medium in a flask and allowed to grow overnight. Following growth, 1 mL of cells were spun down and plated on GADN plates with selected antibiotics. When utilized, X-gal (50 μL of 40 mg/mL 5-bromo-4-chloro-3-indolyl-β-D-galactopyranoside in DMSO) was spotted on and spread across agar plates. An example of a typical strain construct strategy is shown in FIG. 6.

TABLE 2
Gluconacetobacter diazotrophicus strains constructed for this study

| Strain^{a} | Genotype | Vector (Technique) | Reference |
|---|---|---|---|
| Wild-type | Wild-type G. diazotrophicus Pa1 5 (ATCC | None | (1, 2) |
| | 49037) | | |
| GABB019 | nifA::Tet^{R} | Wild-type with pPCRGNIF5 | This study |
| GABB027 | ΔQ-linker | GABB019 with pPCRGNIF3 | This study |
| GABB029 | amtB1::Tet^{R}-lacZ | Wild-type with pGAMTB1 | This study |
| GABB030 | amtB1::Tet^{R}-lacZ with single homologous | GABB029 with pGAMTB2 | This study |
| | recombination of pGAMTB2 | | |
| GABB031 | ΔamtB1 | Recombination of GABB030 | This study |
| GABB032 | amtB2::Tet^{R}-lacZ ΔamtB1 | pGAMTB3 with GABB031 | This study |
| GABB033 | amtB2::Tet^{R}-lacZ with single homologous | pGAMTB4 with GABB032 | This study |
| | recombination of pGAMTB4, ΔamtB1 | | |
| GABB034 | ΔamtB1 ΔamtB2 | Recombination of GABB033 | This study |
| GABB037 | Q-linker::Tet^{R}-lacZ ΔamtB1 ΔamtB2 | pPCRGNIF12 with | This study |
| | | GABB034 | |
| GABB039 | Q-linker::Tet^{R}-lacZ with single homologous | pPCRGNIF14 with | This study |
| | recombination of pPCRGNIF14 ΔamtB1 | GABB037 | |
| | ΔamtB2 | | |
| GABB040 | ΔQ-linker ΔamtB1 ΔamtB2 | Recombination of GABB039 | This study |

^{a}Strains in bold are completed strains containing no antibiotic markers that were used for analysis of elevated nitrogen production.
(1) Bertalan et al., 2009. Complete genome sequence of the sugarcane nitrogen-fixing endophyte Gluconacetobacter diazotrophicus Pa1 5. BMC Genomics 10: 17.
(2) Giongo et al., 2010. Two genome sequences of the same bacterial strain, Gluconacetobacter diazotrophicus Pa1 5, suggest a new standard in genome sequence submission. Stand Genomic Sci 2: 309-317.

TABLE 3
Plasmids

| Plasmid | Relevant Genes(s) and Approach | Parent Vector | Reference |
|---|---|---|---|
| pPCRERIN2 | Cloned amtB1 and flanking regions from G. | pBB053 | (1) |
| | diazotrophicus into pBB053 | | |
| pPCRERIN5 | Cloned amtB2 and flanking regions from G. | pBB053 | (1) |
| | diazotrophicus into pBB053 | | |
| pPCRERIN7 | Plasmid containing two DNA segments flanking regions | pPCRERIN2 | This study |
| | for amtB1 in G. diazotrophicus | | |
| pPCRERIN10 | Plasmid containing two DNA segments flanking regions | pPCRERIN5 | This study |
| | for amtB2 in G. diazotrophicus | | |
| pPCRSACB20 | Moved kanamycin cassette into plasmid containing sacB | pPCRSACB7 and | (1) |
| | gene to construct new cassette with both genes | pPCRKAN4 | |
| pPCRSACB28 | Made various modifications to reorganize sacB gene | pPCRSACB20 | This study |
| | behind the kanamycin resistant gene as a single cassette for | | |
| | simply transfer to other constructs | | |
| pLACZF19 | Moved lacZ gene from pLACZF12 downstream of the | pLACZF12 and | (2, 3) |
| | tetracycline resistant gene in pBBTET6 to create cassette | pPCRTET6 | |
| | containing tetracycline resistance and lacZ | | |
| pGAMTB1 | Plasmid containing the lacZ and tetracycline resistance | pPCRERIN7 and | This study |
| | cassette from pLACZF19 inserted between the flanking | pLACZF19 | |
| | regions of amtB1 | | |
| pGAMTB2 | Plasmid containing the sacB and kanamycin resistance | pPCRERIN7 and | This study |
| | cassette from pPCRSACB28 inserted outside of the second | pPCRSACB28 | |
| | flank for amtB1 | | |
| pGAMTB3 | Plasmid containing the lacZ and tetracycline resistance | pPCRERIN10 and | This study |
| | cassette from pLACZF19 inserted between the flanking | pLACZF19 | |
| | regions of amtB2 | | |
| pGAMTB4 | Plasmid containing the sacB and kanamycin resistance | pPCRERIN10 and | This study |
| | cassette from pPCRSACB28 inserted outside of the second | pPCRSACB28 | |
| | flank for amtB2 | | |
| pPCRGNIF1 | Cloned nifA Q-linker and flanking regions from G. | pBB053 | (1) |
| | diazotrophicus into pBB053 | | |
| pPCRGNIF2 | Plasmid containing two DNA segments flanking Q-linker | pPCRGNIF1 | This study |
| | of nifA in G. diazotrophicus | | |
| pPCRGNIF3 | Performed PCR to remove Q-linker region from nifA in | pPCRGNIF1 | This study |
| | DNA segment from G. diazotrophicus | | |
| pPCRGNIF5 | Plasmid containing tetracycline selection marker from | pPCRGNIF2 and | (3) |
| | pBBTET6 inserted between flanking regions of | pBBTET6 | |
| | pPCRGNIF2 | | |
| pPCRGNIF12 | Plasmid containing the lacZ and tetracycline resistance | pPCRGNIF2 and | This study |
| | cassette from pLACZF19 inserted between the flanking | pLACZF19 | |
| | regions of the Q-linker for nifA | | |
| pPCRGNIF14 | Plasmid containing the sacB and kanamycin resistance | pPCRGNIF3 and | This study |
| | cassette from pPCRSACB28 inserted outside of modified | pPCRSACB28 | |
| | DNA segment containing nifA | | |

(1) Lenneman et al., 2013. Fatty Alcohols for Wax Esters in Marinobacter aquaeolei VT8: Two Optional Routes in the Wax Biosynthesis Pathway. Appl Environ Microbiol 79: 7055-7062.
(2) Barney et al., 2015. Gene deletions resulting in increased nitrogen release by Azotobacter vinelandii: application of a novel nitrogen biosensor. Appl Environ Microbiol 81: 4316-4328.
(3) Schwister et al., 2022. Gluconacetobacter diazotrophicus Gene Fitness during Diazotrophic Growth. Appl Environ Microbiol 88: 1-15.

TABLE 4
Primers

| Primer | Sequence (5′ to 3′) | Purpose | SEQ ID NO: |
|---|---|---|---|
| BBP1747 | CACATGTTCT TTCCTGCGTT ATCCC | Conformation of lacZ insertions | 8 |
| BBP2161 | CGCCTAGCTT CCTGCTGAAC ATC | Conformation of sacB insertions | 9 |
| BBP3158 | NNNGAATTCG TGCTGCCAGC | Cloning amtB1 and flanking | 10 |
| | CCATAAATCA GG | regions | |
| BBP3159 | NNNTCTAGAC GACTCGCTGG | Cloning amtB1 and flanking | 11 |
| | AATATTTGGG CGACAC | regions | |
| BBP3160 | NNNGGATCCG AATTCTGTGC | Delete amtB1 from plasmid | 12 |
| | ATATGGCGTC TTCTCCCGTC TCGC | | |
| BBP3161 | NNNGGATCCC TGATCCATTT | Delete amtB1 from plasmid | 13 |
| | CACCGAACAA CAGACAGG | | |
| BBP3184 | NNNGAATTCC TTTTTTATTT | Cloning amtB2 and flanking | 14 |
| | CACAATCGAT GCAACCAGCG TTTCC | regions | |
| BBP3185 | NNNAAGCTTC TTCGTCAGCC | Cloning amtB2 and flanking | 15 |
| | ATGGCAGGTG GGTATC | regions | |
| BBP3164 | NNNGGATCCG AATTCGTTCA | Delete amtB2 from plasmid | 16 |
| | TATGGCAATC TCCCCGGTTT | | |
| | CATTCGTACG GATAC | | |
| BBP3165 | NNNGGATCCT GATCGTTCCG | Delete amtB2 from plasmid | 17 |
| | GAAACGGGC | | |
| BBP3188 | GATCGTCTCA TGGGCACTCA GTGAC | Conformation of amtB1 | 18 |
| | | manipulations | |
| BBP3189 | GTGTATCAGA CCGTTCCGCT GCAG | Conformation of amtB1 | 19 |
| | | manipulations | |
| BBP3192 | GAATGACGAG ACGTACAAGG | Conformation of amtB2 | 20 |
| | TCGCCATGAC | manipulations | |
| BBP3193 | CGATAGATTT CGCCGCGATA | Conformation of amtB2 | 21 |
| | ATTGAAGG | manipulations | |
| BBP3269 | NNNTCTAGAC TGGTGAGGTG | Cloning Q-linker and flanking | 22 |
| | GAGGAGGCAA GTAG | regions of nifA | |
| BBP3270 | GGTCAGCGAC GTCCGGTTGG TTTCG | Cloning Q-linker and flanking | 23 |
| | | regions of nifA | |
| BBP3271 | CAGGTTTTCC CGGTCCCGCT GCAC | Removal of Q-linker | 24 |
| BBP3272 | GCGCCGGTTG CCGATGGCGG | Removal of Q-linker | 25 |
| BBP3273 | NNNGGATCCC AGGTTTTCCC | Deletion of Q-linker region | 26 |
| | GGTCCCGCTG CAC | | |
| BBP3274 | NNNGGATCCG CGCCGGTTGC | Deletion of Q-linker region | 27 |
| | CGATGGCGG | | |

Algal and G. diazotrophicus Co-Culture Growth

Following confirmation of the various strains constructed as part of this work, G. diazotrophicus strains were tested for elevated nitrogen production. Elevated nitrogen production was screened by growing Chlorella sorokiniana UTEX 1602 (Arriola et al., 2017. Plant J 93:566-586) in the presence of G. diazotrophicus without any supplemented nitrogen present in the growth medium. A similar method has been previously applied where Azotobacter vinelandii was grown with C. sorokiniana (Barney et al., 2015. Appl Environ Microbiol 81:4316-4328).

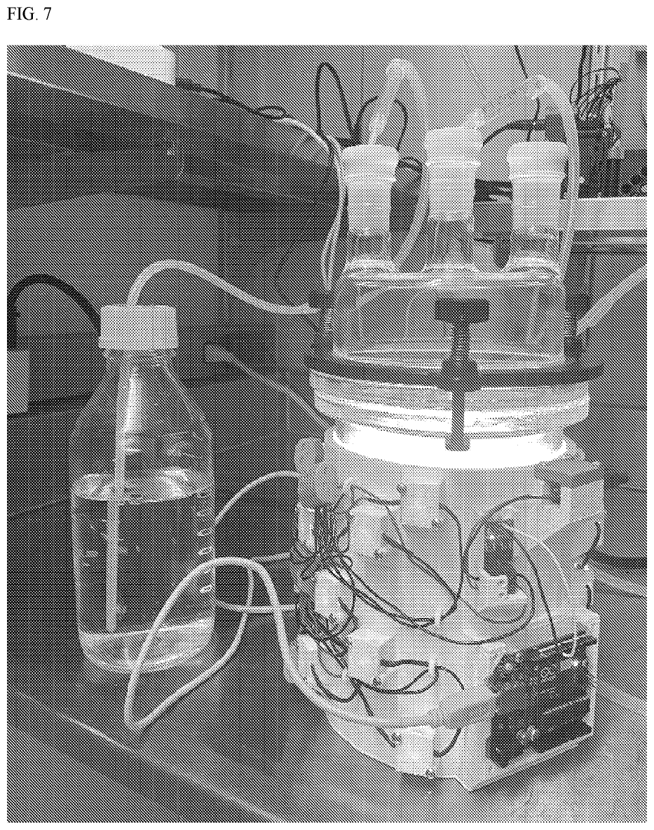

To culture G. diazotrophicus and C. sorokiniana together, cells were first grown separately on their respective medium. After three days of growth on GADN plates, 20 μL (⅓ loop) of G. diazotrophicus was suspended in 0.5 mL of distilled water. C. sorokiniana cells were grown on Freshwater plates for one week, and then a similar quantity of cells were scraped and washed twice before resuspending in 0.5 mL of distilled water. Cells of each culture were diluted 20-fold in distilled water and 50 μL was spotted onto solid agar and allowed to absorb. The spots were grown in a micro-aerobic chamber at 5% oxygen in a 95% nitrogen background. The growth apparatus is shown in FIG. 7. Gases are maintained at a constant flow through mass flow controllers and exhaust gas is bubbled through a flask to create a positive pressure barrier between the external atmosphere.

### Ammonium Quantification of Individual Strains

To measure the accumulation of ammonium, G. diazotrophicus strains were first inoculated from fresh GADN plates into 125 mL Erlenmeyer flasks containing 50 mL of GADN medium and grown to an OD at 600 nm of ˜2.0 to 2.5. Then 1 mL of culture was removed and the cells pelleted at 12,000×g for one minute, and the supernatant removed. The pellet was resuspended in 1 mL of the modified Burk's medium listed below and transferred to a new 125 mL Erlenmeyer flask containing 50 mL of the modified Burk's medium. The modified Burk's (B) medium contained 20 g sucrose, 200 mg MgSO₄·7H₂O, 90 mg CaCl₂)·2H₂O, 0.5 mg Na₂MoO₄·2H₂O, 10 mg FeSO₄·7H₂O, 200 mg KH₂PO₄ and 800 mg K₂HPO₄ in 1 L dH₂O; supplemented with 35 mM sodium citrate and 1.25 mM asparagine and adjusted to pH 6.8 with HCl or NaOH. The asparagine concentration was selected to allow the culture to reach on OD₆₀₀ of approximately 0.5, at which point the culture was able to grow diazotrophically under fully aerobic conditions at 28° C. and 180 rpm and remain in a planktonic state of growth. Flasks were sampled daily for optical density and ammonium quantification. Ammonium was quantified using the o-phthalaldehyde assay protocol as previously described (Barney et al., 2015. Appl Environ Microbiol 81:4316-4328; Barney et al., 2004. J Biol Chem 279:53621-53624). For high ammonium concentrations (>100 μM), a spectrophotometric approach was employed for quantification using a spectrophotometer (Cary 50, Agilent Technologies, Inc., Santa Clara, CA), while for low ammonium concentrations (<100 μM), ammonium was quantified by fluorescence using a FluoroMax+ fluorometer (Horiba Ltd., Kyoto, Japan).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Generally, a first gene is considered “homologous” to a second gene based on known similarities in gene sequence, gene product (e.g., protein) sequence. It is possible to identify functionally equivalent or homologous genes and gene products on the basis of sequence alignment and/or molecular modelling. As used herein, a gene “homologous” to another gene may be functionally homologous, meaning that the gene product is known to have a similar function within an organism, regardless of whether the sequences of each are homologous. For example, G. diazotrophicus amtB may be considered homologous to any other gene encoding a gene product that transports ammonium into the cell and transports ammonia out of the cell. Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1-Aminomethyl transferase-gcvT (Gdia_0534)
MTQPLLRTPL HDLHLSLGAR MVPFGGYEMP VQYRAGVMAE HQHTRAAAGL FDVSHMGQIR
LRARSGRVED AALALERLVP ADILSLKPGR QRYALLTNEQ GGIIDDLMVS RVGDTLLLVV
NAACKDADLA HITAALDDAC IVESLPDRAL IALQGPLAGA ALARLAPASA DMRFMDVAEF
DVAGVPCIVS RSGYTGEDGF ELGMESGGTV RVAEALLAQA EVEPVGLGAR DSLRLEAGLC
LYGSDIGPDT TPVEGALEWS IQKSRRAGGA RAGGFPGADI ILARIQDGAA RRRVGIAVDG
RAPVRGGARL FADAEGHRPV GHVTSGAFGP TAGAPVAMGY VDIAHAATGT ALFAELRGKY
VPVTVAALPF VAPGFKR
SEQ ID NO: 2-NAD+ synthase-nadE (Gdia_1103)
MDVKRAWSGD PLQPGDRIVA DFRSLYRHGF ARVAGCTLPV ALADPATNVA RMADMVRACH
ADGVALAVFP ELGVSGYTIE DLRQQDVLLD GVGAALAALA AATADLVPVV VAGAPLRHGD
ALYNCAVILH RGTVLGVVPK SYLPNYREFY EARQFAPGAG LRGQTIHVAG QTAPFGTDLL
FEAEDVPGLV IAIEICEDLW VPAPPSTDAA LAGATVIANP SASDITVGKA DTRDLLCRSQ
SARALCAYVY AAAGEGESTT DLAWDGQVSV YENGALLAET VRFPQGPNRA TADIDLDLLR
QERARMGSFA DNRAARGLHA TGGDTGWRRV GFALAPPPGD LGLMRRIERF PFVPADPARL
EQDCYEAWTI QVSALKQRLQ ATGTRRMVIG VSGGLDSTQA LLVAVRAADE LGLGRDAVLA
YTMPGFGTSA GTQSNAMALM QALGVTAAEL DIRPTARMML EQMGHPFASG VPQYDVTFEN
VQAGLRTDFL FRLANQHGGI VIGTGDLSEL ALGWCTYGVG DQMSHYNVNA GLPKTLIQHL
IRWVISARRV DDDAAGVLAS ILDTEISPEL VPAGEDQALQ STEERIGPYA LQDFTLFYVL
RHGFRPSRIA FMAEIAWKDA GIGAWPPGFP ADRRVEYDLP TIRHWLSVFL TRFFGFSQFK
RSAMPNGPKV VAGGALSPRG DWRAPSDGNA RLWLEELERN VP
SEQ ID NO: 3-histidine ammonia lyase-hutH (Gdia_1458)
MMTDSLILTP GTLSLADLRR LAFSAPSVTL AAGVRDTLAQ AARSVDRIVA DGRPVYGVNT
GFGKLARTRI ADANLRDLQR NLVLSHAAGI GAPMDDRTVR LILLLKANGL ARGHSGVRPE
IVDLLLEMGN RGVLPVIPQK GSVGASGDLA PLAHMTAVLI GAGQARVDGR VLPGDQALRA
VGLAPVELGP KEGLALLNGT QASTALALVA LFDAERVFQA ALVTGALTLD AARGTDAPFD
PRLHALRGQK GQIECAALYR ALMADSAIRA SHREDDERVQ DPYCLRCQPQ VMGACLDSLR
HAASVLLIEA NAVSDNPIHF AETDEMLSGG NFHAEPVAIA ADLMAIAVSE VGAIAERRLA
LLVDAQMSGL PPFLVRDSGV NSGFMIAQVT AAALASENKT LAHPASIDSL PTSANQEDHV
SMATFAARRV GDIVANVRDI VAIEYLAAVQ GLDFLAPLQT SRPLAGAAAA LRARVPFYDR
DRIFTPDIEA ARDLIADGVP TLLPGIADVL PRLEV
SEQ ID NO: 4-LysR family transcription regulator-dadA (Gdia_2257)
MDLKSLEAVL WIDRIGGFKA AAEALRMTQP AISIRVSQLE AMLETRIFHR SGKRVVPTPV
GVTLIHYAER ILQLRDEALL TIKGHNEENG ILRLGTVETT AHTWLAQLLC RIDEKYPNIT
VDLDIGISDD IQRKVAKGML DVGLFMGPVN SPMMIETPVC SFEVALLAHR TVVGQRGGRT
DLGSLPALPI MTFSRNTVPH SMLHRLLGRE GGSHHRVHAM SSVPTIVSML LSGAGLALLP
PDVVREHLDS GALCRLDVGQ ALPPLNCVAG RLMKYSSGLV EDVVAMARNV AQSAGA
SEQ ID NO: 5-ammonium transporter (Gdia_0598)
MLVSTALVLM MTVPGLALFY GGMVRKKNVL ATLMQSFAIC CIVTIVWMVA GYSLAFGTGS
PYIGDLSRFM LNGIGAQISK GSDVGFTLGL GSANATVMTI PESVFMMFQM TFAIITPALI
TGAFAERMKF SALCVFTILW SLLVYAPVAH WVWSPLGWVA GFGAIDFAGG TVVHINAGVA
GLVTALVVGK RQGYGQDDMS PFNLTYAVIG ASLLWVGWFG FNAGSAVGAN GRAGMAMATT
QIASAAAGVA WMLAEWARTG KPTVLGIISG AVGGLVAITP AAGFVLPGGA LIIGLLAGAV
CYWTATTMKH MLGYDDSLDA FGVHGIGGIL GALLTGVLAY GPLSATDANP AGVVGSFAQL
VTQAKAVGVT IVWCGVVTFI LLKIVDLAIG LRVRSEDEIE GLDMTQHGER IN
SEQ ID NO: 6-ammonium transporter (Gdia_1303)
MLTSTALVLM MTVPGLALFY GGMVRKKNVL ATLMQSFAIC CIITVLWMVA GYSLTFGTGS
PYIGDLSRFM LNGIGAQISK GSDVGFTLGL GSANATVMTI PESVFMMFQM TFAIITPALI
AGSFAERMKF SALCVFTILW SLLVYAPIAH WVWSPLGWVA GFGAVDFAGG TVVHINAGIA
GLVTALVLGK RQGYGQDDMS PFNLTYAVIG ASLLWVGWFG FNAGSAVGSN GRAGMAMATT
QIATAAAGLS WMLAEWARTG KPTVLGIISG AVAGLVAITP AAGFVLPGGA LVIGLITGAV
CYFAATSLKH MLGYDDSLDA FGVHGIGGIL GALLTGVLAY GPLSATDANP AGVVGSFAQF
VTQAKAVGVT IVWCGVVTFI LLKIVDLAIG LRVTTEQEQQ GLDMSLHGEK IS
SEQ ID NO: 7-D-amino ac id dehydrogenase (Gdia_1301)
MKVIVLGAGV IGVTSAWYLA KLGHEVEVVD RQPAAAMETS FANAGQVSPG YSTPWAMPGL
PRKALGWMLQ KHSPLVIRAR IDFAMFRWMT QLLTNCTEHA YDVNKARMLR IAEYSRDCLT
ALREETGITY DDRQRGLIQL FRTDAQLEHA HEDMRLLAES EVPHELLDVA AIVRREPGLA
HAQHLLKGGL FLPGDESGDA HMFTQRLAQK AEELGVTFHY ETGIEGLDAS ASEILGVRTS
TGRMTGDAYV VALGSYSPLL LRPMGIRLPV YPVKGYSLTV PLTDPDRAPV STVNDETYKV
AMTRLGDRIR IGGTAELTGY DLRLSPDRRE TLELSFSDLF GGGDLGRATY WTGLRPNTPD
GTPVVGPSGR FRNLWLNTGH GTLGWTMACG SGHMLADLIA GRRPNIPHLD LSIDRYAS
SEQ ID NO: 28
DRENLLHDSG LAQPAAPVAD
SEQ ID NO: 29
DRENLAPVAD

1. A genetically modified endophytic diazotrophic microbe.

2. The microbe of claim 1, wherein the microbe is genetically modified to secrete a nitrogen-containing compound in an amount greater than a comparable non-genetically modified microbe.

3. The microbe of claim 1, wherein the microbe does not comprise an undesirable exogenous gene.

4. The microbe of claim 1, wherein the microbe does not comprise a selection marker.

5. The microbe of claim 1, wherein the genetic modification comprises a deletion or disruption of a gene involved in import or export of nitrogen-containing compounds.

6. The microbe of claim 5, wherein the genetic modification comprises a deletion or disruption of a gene encoding a gene product that transports ammonium into the cell, recycles lost ammonium back into the cell, and/or transports ammonia out of the cell.

7. The microbe of claim 6, wherein the genetic modification comprises a deletion or disruption of at least a portion of a gene homologous to G. diazotrophicus amtB.

8. (canceled)

9. (canceled)

10. The microbe of claim 6, wherein the genetic modification comprises a deletion or disruption of at least a portion of a gene homologous to G. diazotrophicus NifA.

11. (canceled)

12. The microbe of claim 5, wherein the genetic modification comprises a clean deletion.

13. The microbe of claim 1, wherein the amount of secreted nitrogen-containing compound is effective to support the growth of a non-diazotroph in co-culture.

14. The microbe of claim 1, wherein the nitrogen-containing compound comprises ammonia or ammonium.

15. The microbe of claim 1, wherein the microbe is derived from a bacterium of the class Alphaprotobacteria.

16. The microbe of claim 1, wherein the microbe is derived from a bacterium of the phylum Pseudomonadota.

17. The microbe of claim 1, wherein the microbe is derived from Gluconacetobacter diazotrophicus.

18. The microbe of claim 1, wherein the microbe is derived from Pseudomonas stutzeri, Rhodobacter sphaeroides, Azospirillum sp., Azoarcus sp., Herbaspirillum sp., Klebsiella sp., or Burkholderia sp.

19. The microbe of claim 1, wherein the microbe fixes nitrogen micro-aerobically.

20. The microbe of claim 1, wherein the microbe fixes nitrogen anaerobically.

21. The microbe of claim 1, wherein the microbe fixes nitrogen aerobically.

22. A method of increasing growth of an organism, the method comprising co-culturing the organism and the microbe of claim 1 in an amount effective to increase the growth of the organism compared to growth of the organism in the absence of the diazotrophic microbe.

23. The method of claim 22, wherein increasing the growth of the organism comprises increasing the growth rate of the organism.

24. The method of claim 22, wherein increasing the growth of the organism comprises increasing the cell density of the organism.

25. The method of claim 22, wherein increasing the growth of the organism comprises increasing the crop yield of the organism.
